(19) United States
(12) Patent Application Publication
(10) Pub. No.: US 2005/0147963 A1
(43) Pub. Date: Jul. 7, 2005

(54) Composite organic-inorganic nanoparticles and methods for use thereof

(75) Inventors: Xing Su, Cupertino, CA (US); Jingwu Zhang, Santa Clara, CA (US); Lei Sun, Santa Clara, CA (US); Andrew A. Berlin, San Jose, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(73) Assignee: Intel Corporation, Santa Clara, CA (US)

(21) Appl. No.: 10/748,336

(22) Filed: Dec. 29, 2003

(51) Int. Cl. C12Q 1/70; C12Q 1/68; C12M 1/34; G01N 33/543

(52) U.S. Cl. 435/5; 435/6; 435/287.2; 436/523

(57) ABSTRACT

Composite organic-inorganic nanoparticles (COIN) are provided that produce surface-enhanced Raman signals when excited by a laser. The nanoparticles include metallic colloids and a Raman-active organic compound. The metal required for achieving a suitable SERS signal is inherent in the nanoparticle, and a wide variety of Raman-active organic compounds can be incorporated into the particle. Indeed, a large number of unique Raman signatures can be created by employing nanoparticles containing Raman-active organic compounds of different structures, mixtures, and ratios. Thus, nanoparticles and methods described herein are useful for the simultaneous detection of many analytes in a mixture, resulting in rapid qualitative analysis of a mixture. In addition, since many Raman-active organic compounds can be incorporated into a single nanoparticle, the SERS signal from a single COIN particle is strong relative to SERS signals obtained from Raman-active materials that do not contain the nanoparticles described herein.

### BACKGROUND OF THE INVENTION

1. Field of the Invention

The invention relates generally to nanoparticles that include metallic colloids and organic compounds, and more specifically to the use of such nanoparticles in analyte detection by surface-enhanced Raman spectroscopy.

2. Background Information

Multiplex reactions are parallel processes that exist naturally in the physical and biological worlds. When this principle is applied to increase efficiencies of biochemical or clinical analyses, the principal challenge is to develop a probe identification system that has distinguishable components for each individual probe in a large probe set. High density DNA chips and microarrays are probe identification systems in which physical positions on a solid surface are used to identify nucleic acid or protein probes. The method of using striped metal bars as nanocodes for probe identification in multiplex assays is based on images of the metal physical structures. Quantum dots are particle-size-dependent fluorescent emitting complexes. These physical structure-based identification systems are, however, constrained by their narrow ranges of physical dimensions. To overcome these restraints, developing a chemical structure-based probe identification system becomes plausible.

In addition, the ability to detect and identify trace quantities of analytes has become increasingly important in virtually every scientific discipline, ranging from part per billion analyses of pollutants in sub-surface water to analysis of cancer treatment drugs in blood serum. Raman spectroscopy is one analytical technique that provides rich optical-spectral information, and surface-enhanced Raman spectroscopy (SERS) has proven to be one of the most sensitive methods for performing quantitative and qualitative analyses. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Among many analytical techniques that can be used for chemical structure analysis, Raman spectroscopy is attractive for its capability in providing rich structure information from a small optically-focused area or detection cavity. Compared to a fluorescent spectrum that normally has a single peak with half peak width of tens of nanometers (quantum dots) to hundreds of nanometers (fluorescent dyes), a Raman spectrum has multiple bonding-structure-related peaks with half peak width of as small as a few nanometers. Furthermore, surface enhanced Raman scattering (SERS) techniques make it possible to obtain a 10⁶ to 10¹⁴ fold Raman signal enhancement, and may even allow for single molecule detection sensitivity. Such huge enhancement factors are attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. It is known that chemical enhancement can also be obtained by placing molecules in a close proximity to the surface in certain orientations. Due to the rich spectral information and sensitivity, Raman signatures have been used as probe identifiers to detect a few attomoles of molecules when SERS method was used to burst the signals of specifically immobilized Raman label molecules, which in fact are the direct analytes of the SERS reaction. The method of attaching metal particles to Raman-label-coated metal particles to obtain SERS-active complexes has also been studied. A recent study demonstrated that SERS signal can be generated after attaching thiol containing dyes to gold particle followed silica coating.

Analyses for numerous chemicals and biochemicals by SERS have been demonstrated using: (1) activated electrodes in electrolytic cells; (2) activated silver and gold colloid reagents; and (3) activated silver and gold substrates. None of the foregoing techniques is capable of providing quantitative measurements, however, and consequently SERS has not gained widespread use. In addition, many biomolecules such as proteins and nucleic acids do not have unique Raman signatures because these types of molecules are generally composed of a limited number of common monomers.

Thus, a need exists for compositions and methods that are useful in expanding the utility of surface-enhanced Raman spectroscopy (SERS).

### BRIEF DESCRIPTION OF THE DRAWINGS

FIG. 1 demonstrates that SERS can be used as an amplification step to detect target molecules “a” and “b”.

FIG. 2 illustrates absorbance data vs. time during a typical COIN synthesis.

FIG. 3 illustrates some electron micrographs of exemplary COIN particles described herein.

FIG. 4 illustrates exemplary SERS spectra produced by nanoparticles described herein.

FIG. 5 illustrates a comparison of single label COIN spectra vs. multilabel COIN spectra.

FIG. 6 illustrates some exemplary applications of the nanoparticles described herein as reporters in immuno-assays.

FIG. 7 illustrates exemplary organic compound-induced metal particle aggregation.

FIG. 8A illustrates the zeta potential of silver particles as a function of 8-aza-adenine concentration. FIG. 8B illustrates a time evolution of aggregate size (z-average) in the presence of 20 μM 8-aza-adenine.

FIG. 9 illustrates a TEM analysis of exemplary silver particles described herein.

FIGS. 10A and 10B illustrate a comparison of SERS spectra with COIN spectra.

FIGS. 11A and 11B illustrate a comparison of Raman activities between SERS and COIN.

FIG. 12 illustrates absorption spectra obtained during a typical COIN synthesis.

FIG. 13 illustrates additional exemplary two-label and three-label COIN signatures.

FIG. 14 illustrates a correlation between scanned Raman signal density and COIN particle density.

FIG. 15 illustrates some exemplary organic compounds used to produce the nanoparticles described herein.

FIG. 16 illustrates a schematic of exemplary microspheres described herein.

FIG. 17 is a flow chart illustrating one method for producing the microspheres described herein (inclusion method).

FIG. 18 illustrates an alternative method for producing micropsheres described herein (soak-in method).

FIG. 19 illustrates another alternative method for producing micropsheres described herein (build-in method).

FIG. 20 illustrates another alternative method for producing micropsheres described herein (build-out method).

### DETAILED DESCRIPTION OF THE INVENTION

Composite organic-inorganic nanoparticles (COIN) and methods for use thereof are provided herein. In one embodiment, there are provided composite organic-inorganic nanoparticles. The nanoparticles include a core and a surface, wherein the core includes a metallic colloid including a first metal and a Raman-active organic compound.

In another embodiment, there are provided methods for producing a composite organic-inorganic nanoparticle having a core and a surface. Such methods can be performed, for example, by reducing metallic ions in the presence of a Raman-active organic compound under conditions suitable to form a metallic colloid, thereby producing a composite organic-inorganic nanoparticle having a core and a surface, wherein the core comprises the metallic colloid and the Raman-active organic compound.

In another embodiment, there are provided methods for detecting an analyte in a sample. Such methods can be performed, for example, by contacting a sample containing an analyte with a nanoparticle including a probe, wherein the probe binds to the analyte; and detecting SERS signals emitted by the nanoparticle, wherein the signals are indicative of the presence of an analyte.

In another embodiment, there is provided a system for detecting an analyte in a sample. The system includes an array including more than one nanoparticle; a sample containing at least one analyte; a Raman spectrometer; and a computer comprising an algorithm for analysis of the sample.

In still another embodiment, there is provided a kit for labeling composite organic-inorganic nanoparticles. The kit includes, for example, a plurality of nanoparticles and a biological agent.

In a further embodiment, there are provided methods for identifying a microorganism. Such methods can be performed, for example, by contacting a sample suspected of containing the microorganism with a plurality of nanoparticles; detecting SERS signals upon contacting the sample with the nanoparticles; and associating the SERS signals from the nanoparticles with the identity of the microorganism.

In yet another embodiment, there are provided methods for identifying an analyte. Such methods can be performed, for example, by contacting a sample suspected of containing the analyte with a plurality of nanoparticles; detecting SERS signals upon contacting the sample with the nanoparticles; and associating the SERS signals from the nanoparticles with the identity of the analyte.

In another embodiment, there are provided methods for producing a composite organic-inorganic nanoparticle having a core and a surface. Such methods can be performed, for example, by vapor-depositing a metal in the presence of a Raman-active organic compound under conditions suitable to form metallic nanoparticles, thereby producing a composite organic-inorganic nanoparticle having a core and a surface, wherein the core includes metallic colloids and the Raman-active organic compound.

In another embodiment, there are provided methods for producing a composite organic-inorganic nanoparticle having a core and a surface. Such methods can be performed, for example, by etching a metal surface in the presence of a Raman-active organic compound under conditions suitable to form metallic nanoparticles, thereby producing a composite organic-inorganic nanoparticle having a core and a surface, wherein the core includes metallic colloids and the Raman-active organic compound.

In one embodiment, there are provided composite organic-inorganic nanoparticles including a core and a surface, wherein the core includes a metallic particle including a first metal and an organic compound. In certain embodiments, the metal particles are colloids. As used herein, the term “colloid” refers to nanometer size metal particles suspending in a liquid, usually water. Typical metals contemplated for use in nanoparticles include, for example, silver, gold, platinum, copper, aluminum, and the like.

As used herein, the term “organic compound” refers to any molecule containing at least one aromatic ring and at least one nitrogen atom. As used herein, “Raman-active organic compound” refers to an organic molecule that produces a unique SERS signature in response to excitation by a laser. A variety of organic compounds, both Raman-active and non-Raman active, are contemplated for use as components in nanoparticles. In certain embodiments, Raman-active organic compounds are polycyclic aromatic or heteroaromatic compounds. Typically the Raman-active compound has a molecular weight less than about 500 Daltons.

In addition, it is understood that these compounds can include fluorescent compounds or non-fluorescent compounds. Exemplary Raman-active organic compounds include, but are not limited to, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, 9-amino-acridine, and the like.

Additional, non-limiting examples of Raman-active organic compounds include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds may be obtained from commercial sources (e.g., Molecular Probes, Eugene, Oreg.).

In certain embodiments, the Raman-active compound is adenine, 4-amino-pyrazolo(3,4-d)pyrimidine, or 2-fluoroadenine. In one embodiment, the Raman-active compound is adenine.

When fluorescent compounds are incorporated into nanoparticles described herein, the compounds include, but are not limited to, dyes, intrinsically fluorescent proteins, lanthanide phosphors, and the like. Dyes include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

The nanoparticles are readily prepared using standard metal colloid chemistry. Invention particles are less than 1 μm in size, and are formed by particle growth in the presence of organic compounds. The preparation of such nanoparticles also takes advantage of the ability of metals to adsorb organic compounds. Indeed, since Raman-active organic compounds are adsorbed onto the metal during formation of the metallic colloids, many Raman-active organic compounds can be incorporated into a nanoparticle without requiring special attachment chemistry.

In certain embodiments, primary COINs (i.e., less than 60 nm) are aggregated to form stable clustered structures that range in size from about 50 nm to 200 nm.

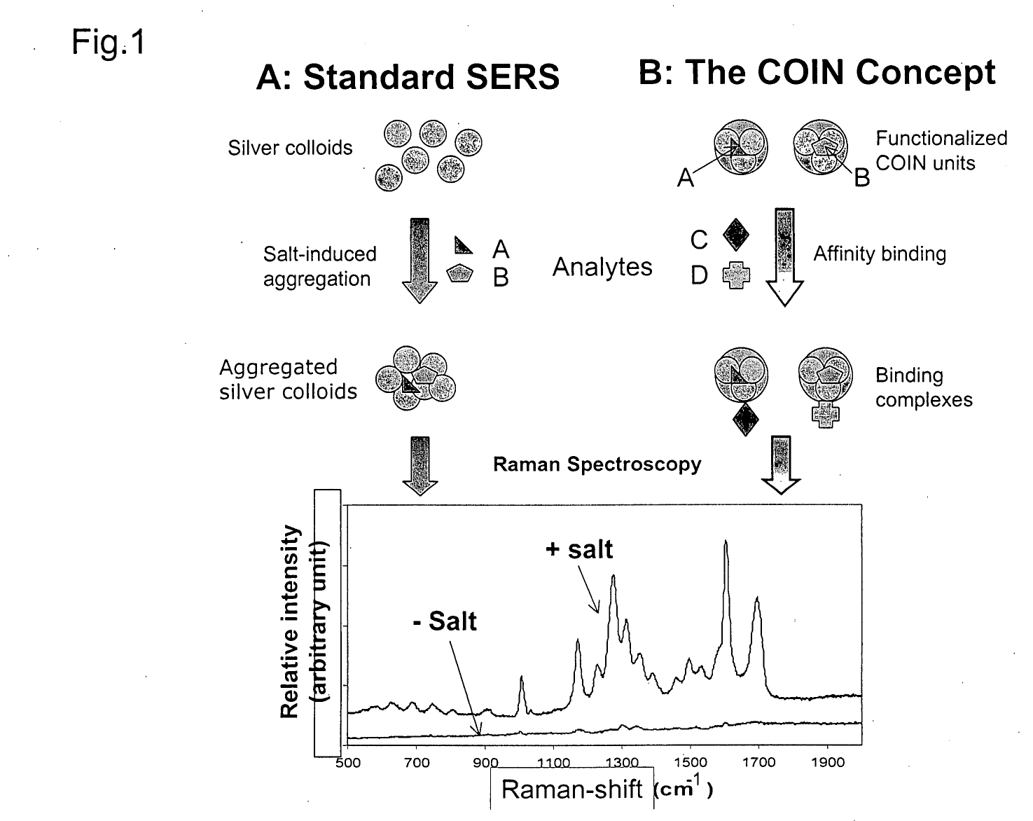

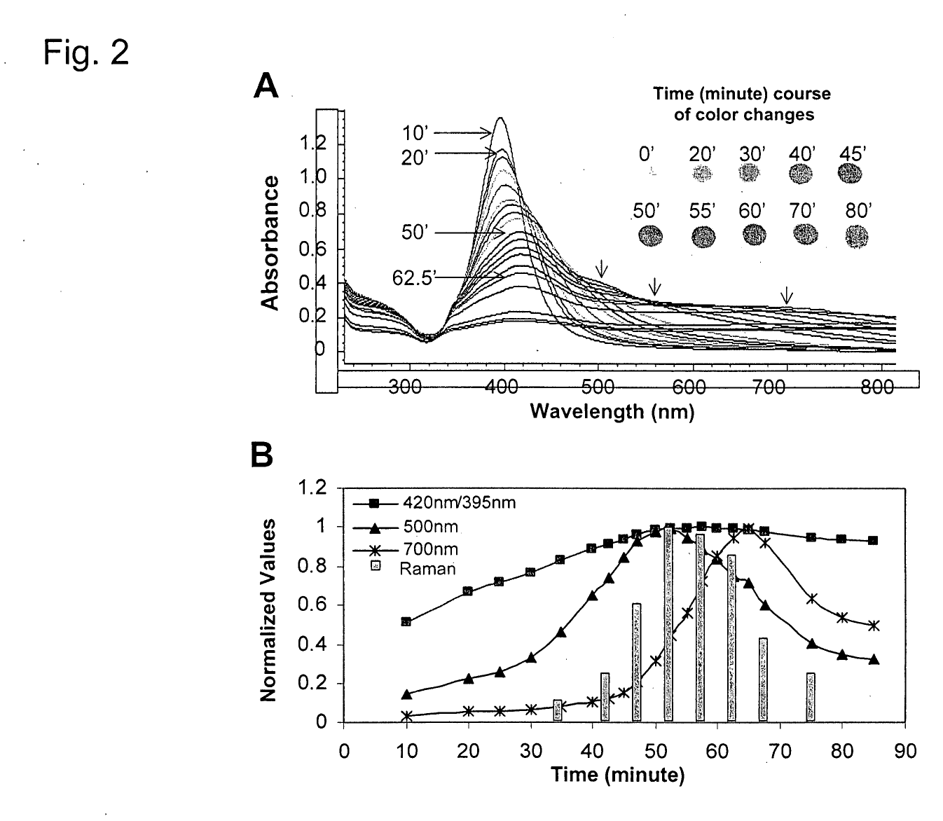

The nanoparticles according to the invention are prepared by a physico-chemical process called Organic Compound Assisted-Metal Fusion (OCAMF). In SERS, the enhancement can be attributed primarily to an increase in the electromagnetic field on curved surfaces of coinage metals. It is also known that chemical enhancement (CE) can be obtained by placing molecules in a close proximity to metal surfaces. Theoretical analysis predicts that electromagnetic enhancement (EME) is particularly strong on rough edges of metal particles. Although individual metal particles have been shown to produce SERS with an enhancement factor as large as 10¹⁴, strongest Raman enhancements, i.e., single molecular detection sensitivity, however, were shown to be associated with colloid clusters formed after salt-induced colloid aggregation. In a typical SERS measurement, the Raman-active molecules are the analytes of the SERS reaction, in which metal atoms or colloids are deposited on or co-aggregated with the analytes. As illustrated in FIG. 1A, SERS can be used as an amplification step to detect target molecules “a” and “b” according to their Raman signatures. The spectra of FIG. 1C show that SERS signal obtained after colloid aggregation induced by salts was at least 10 times stronger than that without salt addition, in which the hardly detectable signals could be resulted from label-induced colloid aggregation.

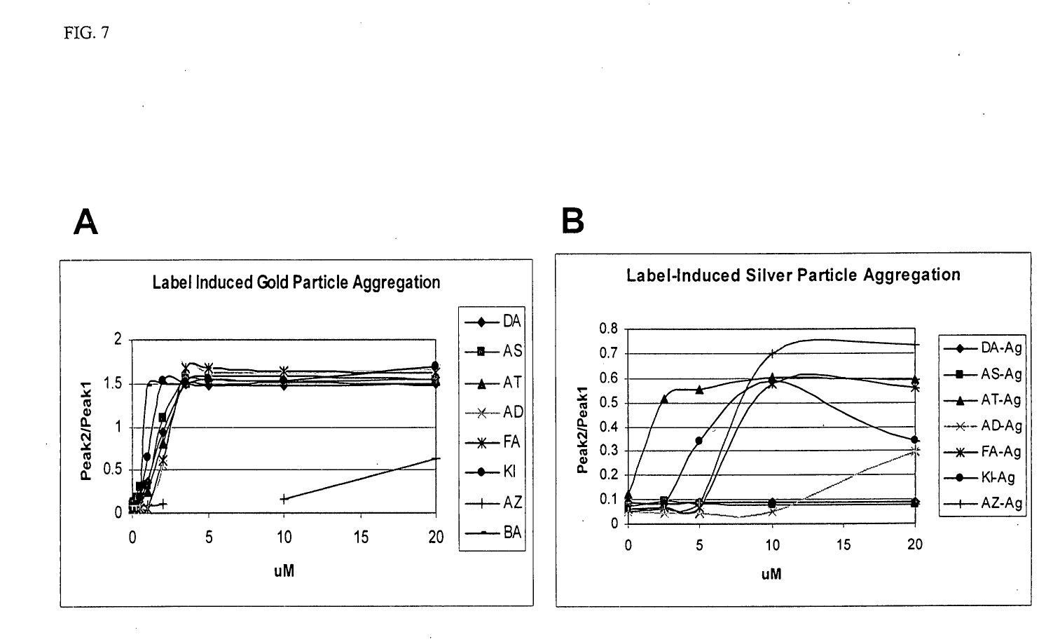

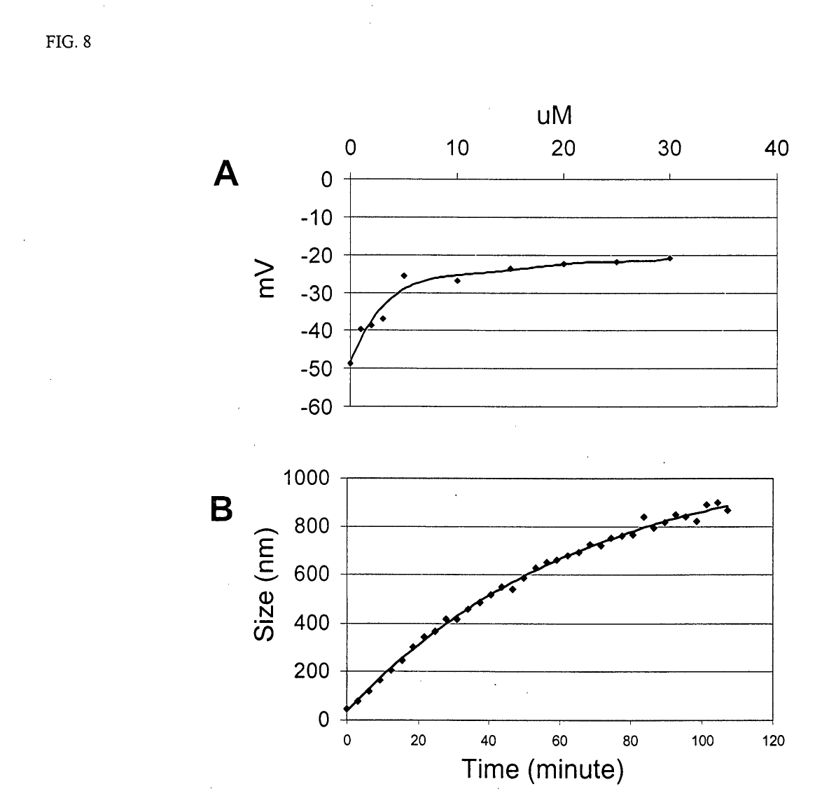

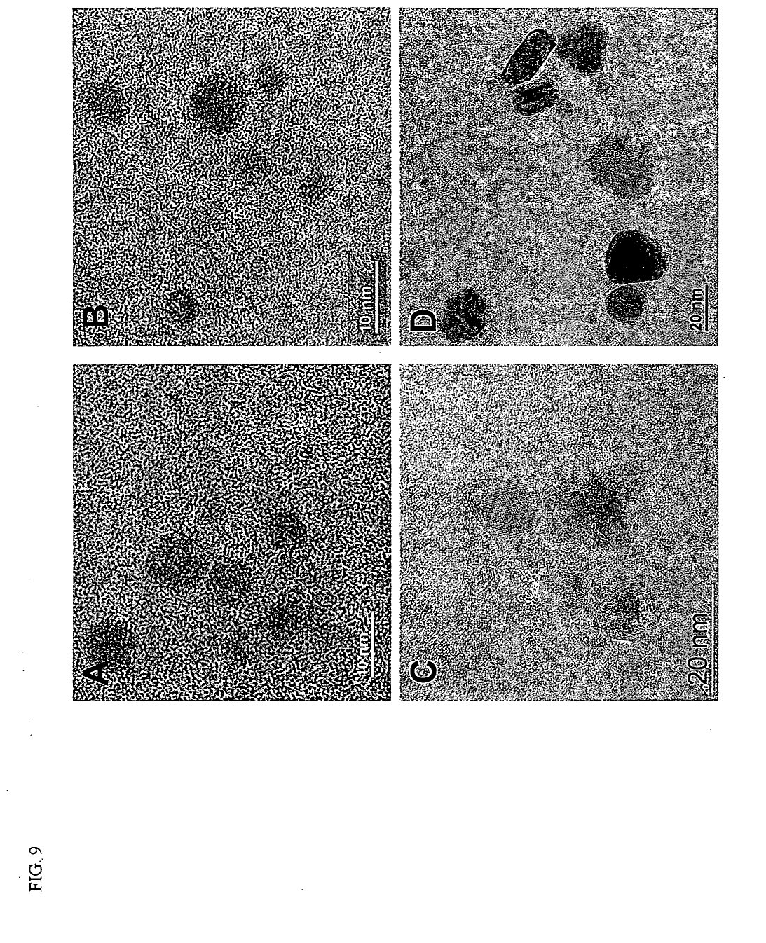

Organic compounds can be absorbed on metal colloids and cause aggregation (FIG. 7) by changing their surface zeta potentials (FIG. 8) and it was found that the aggregated metal colloids fused at elevated temperature (FIG. 9). This chemical phenomenon is called organic compound-assisted metal fusion (OCAMF). It is believed that organic Raman labels could be incorporated into the coalescing metal particles which form stable clusters to produce intrinsically enhanced Raman scattering signals. These composite organic-inorganic nanoparticles (COIN) may be used as reporters for molecular probes. This concept is illustrated in FIG. 1B, in which 2 types of COIN could be made from compounds “a” and “b”, respectively, and then functionalized with specific affinity probes to detect analytes “c” and “d”, respectively. According to the COIN concept, the interaction between the organic Raman label molecules and the metal colloids has mutual benefits. Besides serving as signal sources, the organic molecules promote and stabilize metal particle association that is in favor of EME of SERS. On the other hand, the metal atoms or the metal crystal structures provide spaces to hold and stabilize Raman label molecules, especially those in the cluster junctions.

In general, COINs can be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This type of nanoparticle is referred to as type I COIN. Type I COINs can typically be isolated by membrane filtration. In addition, COINs of different sizes can be enriched by centrifugation.

In a further embodiment of the invention, the nanoparticles include a second metal different from the first metal, wherein the second metal forms a layer overlying the surface of the nanoparticle. To prepare this type of nanoparticle, type I COINs are placed in an aqueous solution containing suitable second metal cations and a reducing agent. The components of the solution are then subject to conditions that reduce the second metallic cations, thereby forming a metallic layer overlying the surface of the nanoparticle. In certain embodiments, the second metal layer includes metals, such as, for example, silver, gold, platinum, aluminum, copper, zinc, iron, and the like. This type of nanoparticle is referred to as type II COINs. Type II COINs can be isolated and or enriched in the same manner as type I COINs. Typically, type I and type II COINs are substantially spherical and range in size from about 20 nm to 60 nm.

In certain embodiments, the metallic layer overlying the surface of the nanoparticle is referred to as a protection layer. This protection layer contributes to aqueous stability of the colloidal nanoparticles. As an alternative to metallic protection layers or in addition to metallic protection layers, COINs can be coated with a layer of silica. If the COINs have already been coated with a metallic layer, such as for example, gold, a silica layer can be attached to the gold layer by vitreophilization of the COINs with, for example, 3-aminopropyltrimethoxysilane (APTMS). Silica deposition is initiated from a supersaturated silica solution, followed by growth of a silica layer by dropwise addition of ammonia and tetraethyl orthosilicate (TEOS). The silica-coated COINs are readily functionalized using standard silica chemistry. In alternative embodiments, titanium oxide or hematite can be used as a protection layer.

In certain other embodiments, nanoparticles include an organic layer overlying the metal layer or the silica layer. Typically, these type of nanoparticles are prepared by covalently attaching organic compounds to the surface of the metal layer in type II COINs. Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

An organic layer can also be used to provide colloidal stability and functional groups for further derivatization. The organic layer is optionally crosslinked to form a solid coating. An exemplary organic layer is produced by adsorption of an octylamine modified polyacrylic acid onto COINs, the adsorption being facilitated by the positively charged amine groups. The carboxylic groups of the polymer are then crosslinked with a suitable agent such as lysine, (1,6)-diaminoheptane, and the like. Unreacted carboxylic groups can be used for further derivation. Other functional groups can be also introduced through the modified polyacrylic backbones.

Furthermore, the metal and organic coatings can be overlaid in various combinations to provide desired properties of coated COINs. For example, COINs may be first coated with a gold layer to seal the more reactive silver before applying the adsorption layer, silica or solid organic coatings. Even if the outer layer is porous, the inner gold layer prevents COINs from chemical attack by different reagents in applications. Another example is to apply an adsorption layer on silica or gold layer to provide additional colloidal stability.

In certain other embodiments, each type of nanoparticles can include cores containing magnetic materials, such as, for example, iron oxides, and the like. Magnetic COINs can be handled without centrifugation using commonly available magnetic particle handling systems. Indeed, magnetism can be used as a mechanism for separating COIN particles tagged with particular biological probes.

For use in the detection of biological molecules, the organic layer can include a probe. In certain embodiments, exemplary probes are antibodies, antigens, polynucleotides, oligonucleotides, receptors, ligands, and the like. In some embodiments, the organic layer can include a polynucleotide probe. The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 10 nucleotides in length, usually at least about 15 nucleotides in length, for example between about 15 and about 50 nucleotides in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides.

The covalent bond linking the nucleotides of a polynucleotide generally is a phosphodiester bond. However, the covalent bond also can be any of numerous other bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization is unavoidable, but is acceptable provided that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42 EC (moderate stringency conditions); and 0.1×SSC at about 68 EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

In some embodiments, the organic layer can include an antibody probe. As used herein, the term “antibody” is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of an analyte.

An antibody is associated with the nanoparticles in certain aspects of the invention. The antibody, for example, includes naturally occurring antibodies as well as non-naturally occurring-antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains. These and other methods of making, for example, chimeric, humanized, CDR-grafted, single chain, and bifunctional antibodies are well known to those skilled in the art.

The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for an epitope of an antigen, are included within the definition of an antibody.

In the context of the invention, the term “ligand” denotes a naturally occurring specific binding partner of a receptor, a synthetic specific-binding partner of a receptor, or an appropriate derivative of the natural or synthetic ligands. As one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

In another embodiment, there are provided methods for detecting an analyte in a sample. Such methods can be performed, for example, by contacting a sample containing an analyte with a nanoparticle including a probe, wherein the probe binds to the analyte; and detecting SERS signals emitted by the nanoparticle, wherein the signals are indicative of the presence of an analyte.

By “analyte” is meant any molecule or compound. An analyte can be in the solid, liquid, gaseous or vapor phase. By “gaseous or vapor phase analyte” is meant a molecule or compound that is present, for example, in the headspace of a liquid, in ambient air, in a breath sample, in a gas, or as a contaminant in any of the foregoing. It will be recognized that the physical state of the gas or vapor phase can be changed by pressure, temperature as well as by affecting surface tension of a liquid by the presence of or addition of salts etc.

As indicated above, methods of the present invention, in certain aspects, detect binding of an analyte to a probe. The analyte can be comprised of a member of a specific binding pair (sbp) and may be a ligand, which is monovalent (monoepitopic) or polyvalent (polyepitopic), usually antigenic or haptenic, and is a single compound or plurality of compounds which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. In certain aspects of the invention, the analyte is charged.

A member of a specific binding pair (“sbp member”) is one of two different molecules, having an area on the surface or in a cavity which specifically binds to and is thereby defined as complementary with a particular spatial and polar organization of the other molecule. The members of the specific binding pair are referred to as ligand and receptor (antiligand) or analyte and probe. Therefore, a probe is a molecule that specifically binds an analyte. These will usually be members of an immunological pair such as antigen-antibody, although other specific binding pairs such as biotin-avidin, hormones-hormone receptors, nucleic acid duplexes, IgG-protein A, polynucleotide pairs such as DNA-DNA, DNA-RNA, and the like are not immunological pairs but are included in the invention and the definition of sbp member.

Specific binding is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme—substrate interactions, polynucleotide hybridization interactions, and so forth.

Non-specific binding is non-covalent binding between molecules that is relatively independent of specific surface structures. Non-specific binding may result from several factors including hydrophobic interactions between molecules.

The nanoparticles of the present invention may be used to detect the presence of a particular target analyte, for example, a nucleic acid, oligonucleotide, protein, enzyme, antibody or antigen. The nanoparticles may also be used to screen bioactive agents, i.e. drug candidates, for binding to a particular target or to detect agents like pollutants. As discussed above, any analyte for which a probe moiety, such as a peptide, protein, oligonucleotide or aptamer, may be designed can be used in combination with the disclosed nanoparticles.

The polyvalent ligand analytes will normally be poly(amino acids), i.e., polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the subject invention can be applied will have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

The monoepitopic ligand analytes will generally be from about 100 to 2,000 molecular weight, more usually from 125 to 1,000 molecular weight. The analytes include drugs, metabolites, pesticides, pollutants, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, peptide nucleic acids (PNA), restriction enzymes, activators, repressors, nucleases, polymerases, histones, repair enzymes, chemotherapeutic agents, and the like.

The analyte may be a molecule found directly in a sample such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

The following paragraphs include further details regarding exemplary applications of COIN probes (i.e., composite organic-inorganic nanoparticles (COIN) that include a probe). It will be understood that numerous additional specific examples of applications that utilize COIN probes can be identified using the teachings of the present specification. One of skill in the art will recognize that many interactions between polypeptides and their target molecules can be detected using COIN labeled polypeptides. In one group of exemplary applications, COIN labeled antibodies (i.e. antibodies bound to a COIN nanoparticle) are used to detect interaction of the COIN labeled antibodies with antigens either in solution or on a solid support. It will be understood that such immunoassays can be performed using known methods such as, for example, ELISA assays. Western blotting, or protein arrays, utilizing the COIN-labeled antibody or COIN labeled secondary antibody, in place of a primary or secondary antibody labeled with an enzyme or a radioactive compound. In another example, a COIN labeled enzyme is used to detect interaction of the COIN-labeled enzyme with a substrate.

Another group of exemplary methods uses COIN probes to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide probe is then used to functionalize a COIN particle (i.e. link a COIN particle to an oligonucleotide probe) using methods disclosed herein, to produce a COIN labeled oligonucleotide probe. The COIN labeled oligonucleotide probe is used in a hybridization reaction to detect specific binding of the COIN labeled oligonucleotide probe to a target polynucleotide. For example, the COIN labeled oligonucleotide probe can be used in a Northern blot or a Southern blot reaction. Alternatively, the COIN labeled oligonucleotide probe can be applied to a reaction mixture that includes the target polynucleotide associated with a solid support, to capture the COIN labeled oligonucleotide probe. The captured COIN labeled oligonucleotide probe can then be detected using Raman spectroscopy, with or without first being released from the solid-support. Detection of the specific Raman label on the captured COIN labeled oligonucleotide probe, identifies the nucleotide sequence of the oligonucleotide probe, which in turn provides information regarding the nucleotide sequence of the target polynucleotide.

In another exemplary group of specific applications, a COIN labeled nucleotide is utilized to determine the nucleotide occurrence at a single base variation in a target polynucleotide. These applications include detection of “hot spot” point mutations and identification of the base at single nucleotide polymorphism (“SNP”) sites. For example, an oligonucleotide primer is prepared that hybridizes immediately adjacent to a polymorphic site. The primer, a target polynucleotide that includes the site of the single base variation, and a polymerase are included in an extension reaction mixture. The reaction mixture includes the four chain terminating triphosphates, each with a unique COIN label attached. The extension reaction then proceeds and, in the case of a homozygous SNP, only one of the four chain-terminating nucleotides is added to the end of the primer, thereby generating a COIN labeled elongated primer. The COIN label on the elongated primer is then detected using raman spectroscopy. The identity of the label identifies the nucleotide added at the site of the single base variation, thereby identifying the nucleotide occurrence at the single base variation in the target polynucleotide.

In the methods of the invention, a “sample” includes a wide variety of analytes that can be analyzed using the nanoparticles described herein, so long as the subject analyte is capable of generating SERS signals upon laser irradiation. For example, a sample can be an environmental sample and includes atmospheric air, ambient air, water, sludge, soil, and the like. In addition, a sample can be a biological sample, including, for example, a subject's breath, saliva, blood, urine, feces, various tissues, and the like.

Commercial applications for the invention methods employing the nanoparticles described herein include environmental toxicology and remediation, biomedicine, materials quality control, food and agricultural products monitoring, anaesthetic detection, automobile oil or radiator fluid monitoring, breath alcohol analyzers, hazardous spill identification, explosives detection, fugitive emission identification, medical diagnostics, fish freshness, detection and classification of bacteria and microorganisms both in vitro and in vivo for biomedical uses and medical diagnostic uses, monitoring heavy industrial manufacturing, ambient air monitoring, worker protection, emissions control, product quality testing, leak detection and identification, oil/gas petrochemical applications, combustible gas detection, H₂S monitoring, hazardous leak detection and identification, emergency response and law enforcement applications, illegal substance detection and identification, arson investigation, enclosed space surveying, utility and power applications, emissions monitoring, transformer fault detection, food/beverage/agriculture applications, freshness detection, fruit ripening control, fermentation process monitoring and control applications, flavor composition and identification, product quality and identification, refrigerant and fumigant detection, cosmetic/perfume/fragrance formulation, product quality testing, personal identification, chemical/plastics/pharmaceutical applications, leak detection, solvent recovery effectiveness, perimeter monitoring, product quality testing, hazardous waste site applications, fugitive emission detection and identification, leak detection and identification, perimeter monitoring, transportation, hazardous spill monitoring, refueling operations, shipping container inspection, diesel/gasoline/aviation fuel identification, building/residential natural gas detection, formaldehyde detection, smoke detection, fire detection, automatic ventilation control applications (cooking, smoking, etc.), air intake monitoring, hospital/medical anesthesia & sterilization gas detection, infectious disease detection and breath applications, body fluids analysis, pharmaceutical applications, drug discovery, telesurgery, and the like.

Another application for the sensor-based fluid detection device in engine fluids is an oil/antifreeze monitor, engine diagnostics for air/fuel optimization, diesel fuel quality, volatile organic carbon measurement (VOC), fugitive gases in refineries, food quality, halitosis, soil and water contaminants, air quality monitoring, leak detection, fire safety, chemical weapons identification, use by hazardous material teams, explosive detection, breathalyzers, ethylene oxide detectors and anaesthetics.

In another embodiment, there are provided systems for detecting an analyte in a sample. Such systems include, an array comprising more than one nanoparticle; a sample containing at least one analyte; a Raman spectrometer; and a computer including an algorithm for analysis of the sample.

A variety of analytical techniques can be used to analyze the COIN particles described herein. Such techniques include for example, nuclear magnetic resonance spectroscopy (NMR), photon correlation spectroscopy (PCS), IR, surface plasma resonance (SPR), XPS, scanning probe microscopy (SPM), SEM, TEM, atomic absorption spectroscopy, elemental analysis, UV-vis, fluorescence spectroscopy, and the like.

In the practice of the present invention, the Raman spectrometer can be part of a detection unit designed to detect and quantify nanoparticles of the present invention by Raman spectroscopy. Methods for detection of Raman labeled analytes, for example nucleotides, using Raman spectroscopy are known in the art. (See, e.g., U.S. Pat. Nos. 5,306,403; 6,002,471; 6,174,677). Variations on surface enhanced Raman spectroscopy (SERS), surface enhanced resonance Raman spectroscopy (SERRS) and coherent anti-Stokes Raman spectroscopy (CARS) have been disclosed.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoparticles is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, that includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the raman-active organic compounds of the nanoparticles and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of the nanoparticles of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects of the invention, a system for detecting the nanoparticles of the present invention includes an information processing system. An exemplary information processing system may incorporate a computer that includes a bus for communicating information and a processor for processing information. In one embodiment of the invention, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor may be a Celeron®, an Itanium®, or a Pentium Xeon® processor (Intel Corp., Santa Clara, Calif.). In various other embodiments of the invention, the processor may be based on Intel® architecture, such as Intel® IA-32 or Intel® IA-64 architecture. Alternatively, other processors may be used. The information processing and control system may further comprise any peripheral devices known in the art, such as memory, display, keyboard and/or other devices.

In particular examples, the detection unit can be operably coupled to the information processing system. Data from the detection unit may be processed by the processor and data stored in memory. Data on emission profiles for various raman labels may also be stored in memory. The processor may compare the emission spectra from composite organic-inorganic nanoparticles in the flow path and/or flow-through cell to identify the raman-active organic compound. The processor may analyze the data from the detection unit to determine, for example, the sequence of a polynucleotide bound by a probe of the nanoparticles of the present invention. The information processing system may also perform standard procedures such as subtraction of background signals.

While certain methods of the present invention may be performed under the control of a programmed processor, in alternative embodiments of the invention, the methods may be fully or partially implemented by any programmable or hardcoded logic, such as Field Programmable Gate Arrays (FPGAs), TTL logic, or Application Specific Integrated Circuits (ASICs). Additionally, the disclosed methods may be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages may be used to analyze the data obtained from the detection unit. In alternative embodiments of the invention, data analysis may be performed, using an information processing system and publicly available software packages.

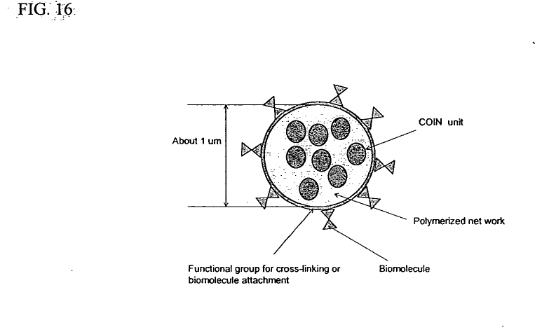

In another embodiment of the invention, there are provided microspheres including polymeric beads and a plurality of invention nanoparticles. Such microspheres produce stronger and more consistent signals. The large microsphere can also provide sufficient surface areas for biomolecule attachment. The structural features are a) a framework formed by polymerized organic compounds; b) multiple COINs embedded in each micro-sized particle; c) providing surface with suitable functional groups for organic molecule attachment (FIG. 16). Several methods for producing microspheres according to this embodiment are set forth below.

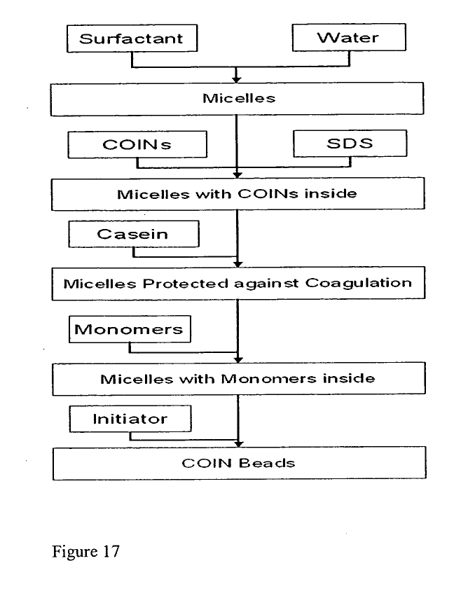

Inclusion method (FIG. 17): This approach employs the well established emulsion polymerization technique for preparing uniform latex microspheres except that COINs are introduced into the micelles before polymerization is initiated. As shown in the flow chart (FIG. 17), this aspect of the involves the following steps: 1) Micelles of desired dimensions are first prepared by homogenization of water with surfactants (e.g. octanol). 2) COINS particles are introduced along with a hydrophobic agent (e.g. SDS). The latter facilitates the transport of COINs into the interior of micelles. 3) Micelles are protected against aggregation with a stabilizing agent (e.g. Casein). 4) Monomers (e.g. styrene or methyl methacrylate) are introduced. 5) Finally, a free radical initiator (e.g. peroxide or persulfate) is used to start the polymerization to produce COIN embedded latex beads.

An important refinement of the above approach is to use COIN particles which have been coated with a solid organic polymer layer. The coating can prevent direct contact between COIN particles in the micelles and in the final product (COIN beads). Furthermore, the number of COINs in each bead can be adjusted by varying the thickness of organic coating.

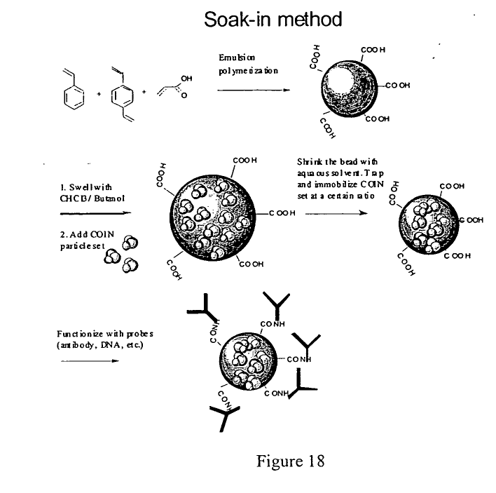

Soak-in method (FIG. 18): Microspheres are obtained first and allowed to contact with COINs that are synthesized separately. Under certain conditions, such as in an organic solvent, the pores of the beads are enlarged enough to allow COINs to diffuse inside. After the liquid phase is changed to an aqueous phase, the COINs are encapsulated. For example, 1) Styrene monomers are co-polymerized with divinylstyrene and acrylic acid to form uniformly-sized beads through emulsion polymerization. 2) The beads are swelled with organic solvents such as chloroform/butanol, and a set of COINs at a certain ratio are introduced so that the COINs diffuse into the swollen bead. 3) The beads are then placed in a non-solvent to shrinks the beads so that the COINs are trapped inside to form stable, uniform COIN-encapsulated beads.

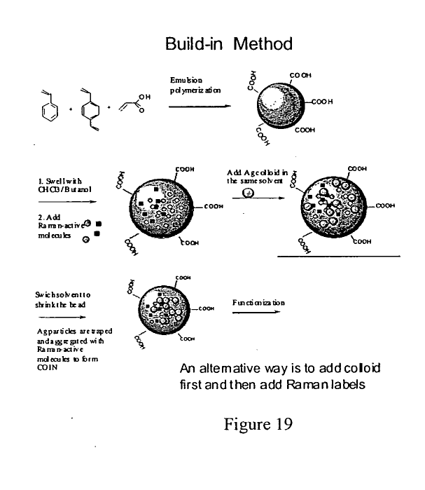

Build-in method (FIG. 19): In this method, microsphere beads are obtained first and are placed in contact with Raman labels and silver colloids in organic solvents. Under this condition, the pores of the beads are enlarged enough to allow the labels and silver colloids to diffuse inside. Then COIN clusters are formed inside the microsphere beads when silver colloid encounters each other in the presence of organic Raman labels. Heat and light can be used to accelerate aggregation and fusion of silver particles. Finally, the liquid phase is changed to aqueous phase, the COINs are encapsulated. For example, 1) Styrene monomers are co-polymerized with divinylstyrene and acrylic acid to form uniformly-sized beads through emulsion polymerization. 2) The beads are then swelled with organic solvents such as chloroform/butanol, and a set of Raman-active molecules (i.e. 8-azaadenine and N-benzoyladenine) at a certain ratio is introduced so that the molecules diffuse into the swollen bead. Ag colloid suspension in the same solvent is then mixed with the beads to form Ag particle-encapsulated beads. 3) The solvent was switched to one that shrinks the beads so that the Raman labels and Ag particles are trapped inside. The process can be controlled so that the Ag particles will contact each other with Raman molecules in the junction, forming COIN inside the beads. When medium size silver colloids such as 60 nm are used, Raman labels are added separately (before or after silver addition) to induce colloid aggregation (formation of COINs) inside the beads, when 1-10 nm colloids are used, the labels can be added together, then light or heat is used to induce the formation of active COINs inside the beads.

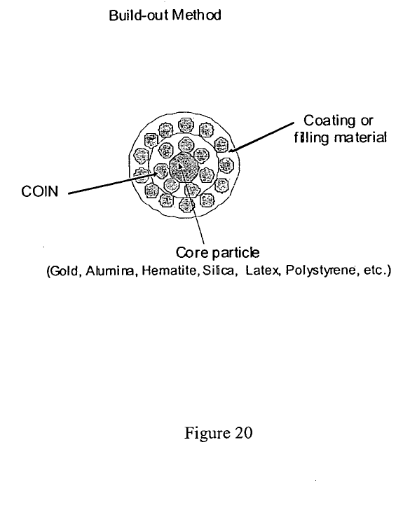

Build-out method (FIG. 20): In this method, a solid core is used first as the support for COIN attachment. The core can be metal (gold and silver) particles, inorganic (alumina, hematite and silica) or organic (polystyrene, latex) particles. Attachment of COINs to the core particle can be induced by electrostatic attraction, van der Waals forces, and/or covalent binding. After the attachment, the assembly can be coated with a polymer to stabilize the structure and at the same time to provide a surface with functional groups. Multiple layers of COINs can be built based on the above procedure. The dimension of COIN beads can be controlled by the size of the core and the number of COIN layers. For example, 1) positively charged Latex particles of 0.5 μm are mixed with negatively charged COINs, 2) coating the Latex-COIN complex with a cross-linkable polymer such as poly-acrylic acid. 3) Cross-linking the polymer coating with linker molecules such as lysine to form an insoluble shell. Remaining (unreacted) carboxylic groups would serve as the functional groups for second layer COIN attachment or probe attachment. Additional functional groups can also be introduced through co-polymerization or during the cross-link process.

The invention will be further understood with reference to the following examples, which are purely exemplary, and should not be taken as limiting the true scope of the present invention as described in the claims.

### EXAMPLES

General Considerations

Chemical reagents: Biological reagents including anti-IL-2 and anti-IL-8 antibodies were purchased from BD Biosciences Inc. The capture antibodies for analytes were monoclonal antibodies generated from mouse, and the detection antibodies were polyclonal antibodies generated from mouse and conjugated with biotin. All other chemicals were purchased, at highest available quality, from Sigma Aldrich Chemical Company (St. Louis, Mo., USA) unless otherwise indicated. Deionized water with resistance of 1 8.2×10⁶ Ohms-cm (Nanopure Infinity, Barnstead) was used for experiments.

Silver seed particle synthesis: Silver nitrate (AgNO₃) and sodium citrate (Na₃Citrate) stock solutions (0.50 M) were filtered twice through 0.2 micron polyamide membrane filters (Schleicher and Schuell, Germany) which were thoroughly rinsed before use. Sodium borohydrate solution (50 mM) was made freshly and used within 2 hours after preparation. Silver seed particles were prepared by rapid addition of 50 mL of Solution A (containing 8.00 mM sodium citrate, 0.60 mM sodium borohydrate and 2.00 mM sodium hydroxide) into 50 mL of Solution B (containing 4.00 mM silver nitrate) under vigorous stirring. Addition of Solution B into solution A led to a more polydispersed suspension. Silver seed suspensions, stored in the dark, were used within one week after preparation. Before use, the suspension was analyzed by Photon Correlation Spectroscopy (PCS, Zetasizer 3000 HS, Malvern) to ensure the intensity-averaged diameter (z-average) was between 10-12 nm with a polydispersity index less than 0.25.

Gold seed synthesis: A household microwave oven (1350 W, Panasonic) was used to prepare gold nanoparticles. More detailed preparation conditions will be published elsewhere. Typically, 40 mL of an aqueous solution containing 0.5 mM HAuCl₄ and 2.0 mM sodium citrate in a glass bottle (100 mL) was heated to boiling in the microwave using the maximum power, followed by a lower power setting to keep the solution gently boiling for 5 min. 2.0 grams of PTFE boiling stones (6 mm, Saint-Gobain A1069103, through VWR) was added to the solution to promote gentle and efficient boiling. The resultant solutions had a rosy red color. Measurements by PCS showed that the gold solutions had a typical z-average of 13 nm with a polydispersity index of <0.04.

### Example 1

### Synthesis of Composite Organic-Inorganic Nanoparticles (COIN)

This example illustrates typical syntheses of the nanoparticles described herein.

Reflux method: To prepare COIN particles with silver seeds, typically, 50 mL silver seed suspension (equivalent to 2.0 mM Ag⁺) was heated to boiling in a reflux system before introducing Raman labels. Silver nitrate stock solution (0.500 M) was then added dropwise or in small aliquots (50-100 μL) to induce the growth and aggregation of silver seed particles. Up to a total of 2.5 mM silver nitrate could be added. The solution was kept boiling until the suspension became very turbid with a dark brown color. At this point, the temperature was lowered quickly by transferring the colloid solution into a glass bottle and then stored it at room temperature. The optimum heating time depended on the nature of Raman labels and amounts of silver nitrate addition. It was found helpful to verify that particles had reached a desired size range (80-100 nm on average) by PCS or UV-Vis spectrophotometer before the heating was arrested. Normally, the dark brown color was the indication of cluster formation and associated Raman activity.

To prepare COIN particles with gold seeds, typically, gold seeds were first prepared from 0.25 mM HAuCl₃ in the presence of Raman labels (e.g., 20 μM 8-aza-adenine). After heating the gold seed solution to boiling, silver nitrate and sodium citrate stock solutions (0.5 M) were added, separately, so that the final gold suspension contained 1.0 mM AgNO₃ and 1.0 mM sodium citrate. Silver chloride precipitate might form immediately after silver nitrate addition but disappeared soon with heating. After boiling an orange-brown color developed and stabilized, an additional aliquot (50-100 ul) of silver nitrate and sodium citrate stock solutions (0.500M each) were added to induce the development of a green color, which was the indication of cluster formation and associated with Raman activity. Note that the 2 procedures produced COINs with different colors, primarily due to the differences in the size of primary particles before cluster formation).

Oven method: COINs could also be prepared conveniently by using a convection oven. Silver seed suspension was mixed with sodium citrate and silver nitrate solutions in a 20 mL glass vial. The final volume of the mixture was typically 10 mL, which contained silver particles (equivalent to 0.5 mM silver ions), 1.0 mM silver nitrate and 2.0 mM sodium citrate (including the portion from the seed suspension). The glass vials were incubated in the oven set at 95° C. for 60 min before being stored at room temperature. A range of label concentrations could be tested at the same time. Batches showing brownish color with turbidity were tested for Raman activity and colloidal stability. Bathes with significant sedimentation (when the label concentrations were too high) were discarded. Occasionally, batches that did not show sufficient turbidity could be kept at room temperature for an extended period of time (up to 3 days) to allow cluster formation. In many cases, suspensions became more turbid over time due to aggregation, and strong Raman activity developed within 24 hours. A stabilizing agent, such as bovine serum albumin (BSA), could be used to stop the aggregation and stabilize the COIN particles.

A similar approach was used to prepare COINs with gold cores. Briefly, 3 mL of gold suspensions (0.5 mM Au⁺⁺⁺) prepared in the presence of Raman labels was mixed with 7 mL of silver citrate solution (containing 5 mM silver nitrate and 5 mM sodium citrate before mixing) in a 20 mL glass vial. The vial was placed in a convection oven heated to 95° C. for 1 hour. Different concentrations of labeled gold seeds could be used simultaneously in order to produce batches with sufficient Raman activities.

It should be noted that a COIN sample can be heterogeneous in terms of size and Raman activity. We typically used centrifugation (200-2,000×g for 5-10 min) or filtration (300 kDa, 1000 kDa or 0.2 micron filters, Pall Life Sciences through VWR) to enrich for particles in the range of 50-100 nm. It is recommended to coat the COIN particles with a protection agent (e.g., BSA, antibody) before enrichment. Some lots of COINs that we prepared (with no further treatment after synthesis) were stable for more than 2 months at room temperature without noticeable changes in physical and chemical properties.

Particle size measurement: The size of silver and gold seed particles as well as COINs was determined by using Photon Correlation Spectroscopy (PCS, Zetasizer 3000 HS or Nano-ZS, Malvern). All measurements were conducted at 25° C. using a He—Ne laser at 633 nm. Samples were diluted with DI water when necessary.

TEM analysis: for transmission electronic microscopic (TEM) analysis, carbon coated Copper grids were used for sample preparation. The sample suspensions were spayed on to the grid using a all-glass nabulizer (Ted Pella). Alternatively, a drop (20 μL) of sample suspension was deposited on the grid. After five minutes, the drop was blotted off with a piece of filter paper. Then the grid was allowed to touch the surface of a DI water drop for a few seconds to remove salts before drying in the air. TEM observation was made by using either JEM 2010 or 2010F with a UHR pole (Japan Electron Optics Laboratories).

SEM analysis: for scanning electronic microscopic (SEM) analysis, COIN particles were examined under a scanning electro microscope (S-4500, Hitachi). The sample preparation procedure was as follows: a small piece of silicon wafer substrate (1×1 cm²) was wet with a drop (20 ul) of poly-L-lysine (0.1%); after 5 min, the substrate was rinsed with deionized water (DI-water) and dried under a stream of nitrogen; a 20 μL of colloidal sample was then deposited on the Poly-L-lysine-coated substrate. The substrate was finally rinsed with DI-water and let dry in air before SEM observation.

Raman spectral analysis: for all SERS and COIN assays in solutions, a Raman spectroscope (Renishaw Transdeucer Systems Ltd., UK) equipped with 514 nm Argon ion laser (50 mW) was used. Typically, a drop (50-200 uL) of a sample was placed on an aluminum surface. The laser beam was focused on the top surface of the sample and photons were collected for 10-20 second. The Raman system normally generated about 600 counts from methanol at 1040 cm⁻¹ for 10 second collection time.

For Raman spectroscopy detection of analyte immobilized on surface, Raman spectra were recorded using a Raman microscope built in-house. This Raman microscope consisted of a water cooled Argon ion laser operating in continuous-wave mode, a dichroic reflector, a holographic notch filter, a grating spectrometer, and a liquid nitrogen cooled CCD camera. The spectroscopy components were coupled with a microscope so that the microscope objective focused the laser beam onto a sample, and collected the back-scattered Raman emission. The laser power at the sample was ˜60 mW. All Raman spectra were collected with 514 nm excitation wavelength.

Absorption spectral analysis: Extinction spectra for Raman labels and colloidal suspensions were recorded by an UV-Vis spectrophotometer (Model 8453, Agilent Technologies).

### Example 2

### Coin Raman Signals are Intrinsic

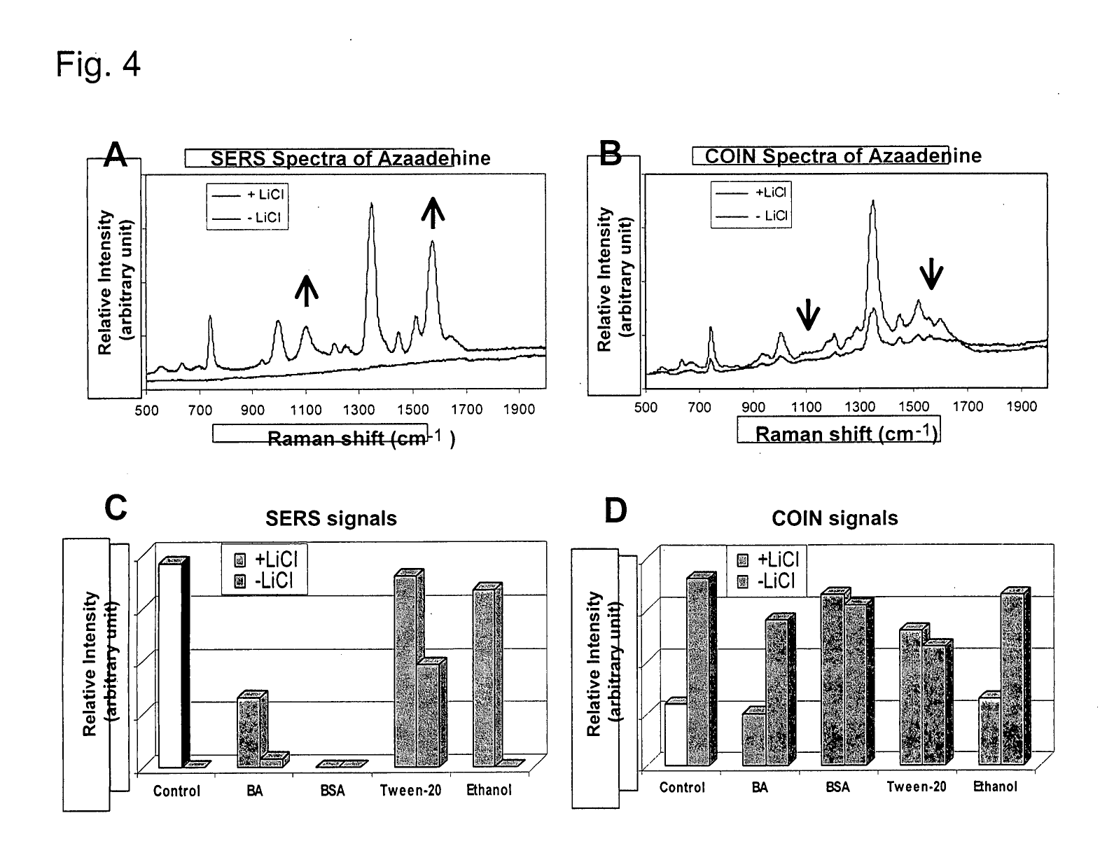

To further validate the COIN concept, Raman activity of standard SERS reactions was compared with Raman activities of COIN. As an example of a standard SERS reaction, a typical Raman spectrum was obtained when 4 μM aza-adenine was mixed with a silver colloid solution and a monovalent salt (FIG. 4A). When the salt was omitted from the reaction, Raman signals were not detected. To the contrary, a strong Raman signal was detected from a COIN sample with no salt added; but the Raman signal was greatly reduced when salt was included (FIG. 4B). The salt-independence of COIN signals suggested that, unlike standard SERS, salt-induced particle aggregation was not required for COIN to produce a Raman signal. Compared to the Raman spectrum of the standard SERS reaction, the peaks at 1100 cm⁻¹ and 1570 cm⁻¹ disappeared almost completely from the Raman spectrum of COIN. Similar phenomena were observed for other Raman labels that were tested (FIG. 8). The disappearance of these peaks indicates that the Raman label molecules in the COIN particles were associated with the metal structures in a way that was different from that found in normal surface adsorption as in SERS.

In a similar test, benzoyl-adenine and aza-adenine was added to a silver colloid solution, and it was found that the aza-adenine signal was suppressed, compared to the control where only 4 μM aza-adenine was used (FIG. 4C). In exactly the same samples, the benzoyl-adenine signal was very strong as expected (FIG. 8A). This could be caused by the binding site competition by the 2 Raman labels. The slightly evaluated signal when salt was not added likely resulted from label-induced colloid aggregation. When the same concentration of benzoyl-adenine was added to COIN (prepared from aza-adenine but no additional aza-adenine was used in the test), the aza-adenine signal was only slightly affected. The aza-adenine signal was suppressed greatly when salt was used (FIG. 4D). At the same time, the benzoyl adenine signal was hardly noticeable (FIG. 8B). This indicated that the COIN particle surfaces have a negligible SERS activity for extra Raman labels.

SERS and COIN were compared under other conditions. FIG. 4C showed that SERS was completely inhibited by 0.5% bovine serum albumin (BSA). To the contrary, the Raman signal for aza-adenine did not change significantly regardless of the presence or absence of salt (FIG. 4D). These results suggest that a) COIN is compatible with proteins and b) a surface coating of COIN may actually stabilize COIN (preventing COIN from aggregation). Tween-20 is a non-ionic surfactant commonly used for biochemical reactions. It was found that it did not inhibit salt-induced SERS reactions and it appeared to promote some SERS activity in the absence of salt (FIG. 4C). When Tween-20 was added to COIN in the presence of salt, no obvious aggregation was noticed and thus COIN signals were not significantly suppressed. However, in the absence of salt, a fraction of the COIN signal was reduced (FIG. 4D). These results could be explained by the fact that while Tween-20 could inhibit salt-induced aggregation, the surfactant itself could cause particle colloid aggregation. SERS reaction in the presence of 30% ethanol (plus salt) enhanced the peak at 1550 cm⁻¹ comparing to ethanol free reactions (FIG. 8G). On the other hand, COIN behaved the same as if it was in water in terms of spectra and relative peak intensities (FIG. 4D and FIG. 8H). The above data indicate that Raman signals detected from COIN are intrinsic.

### Example 3

### A Wide Range of Raman Labels Can Be Incorporated into COIN

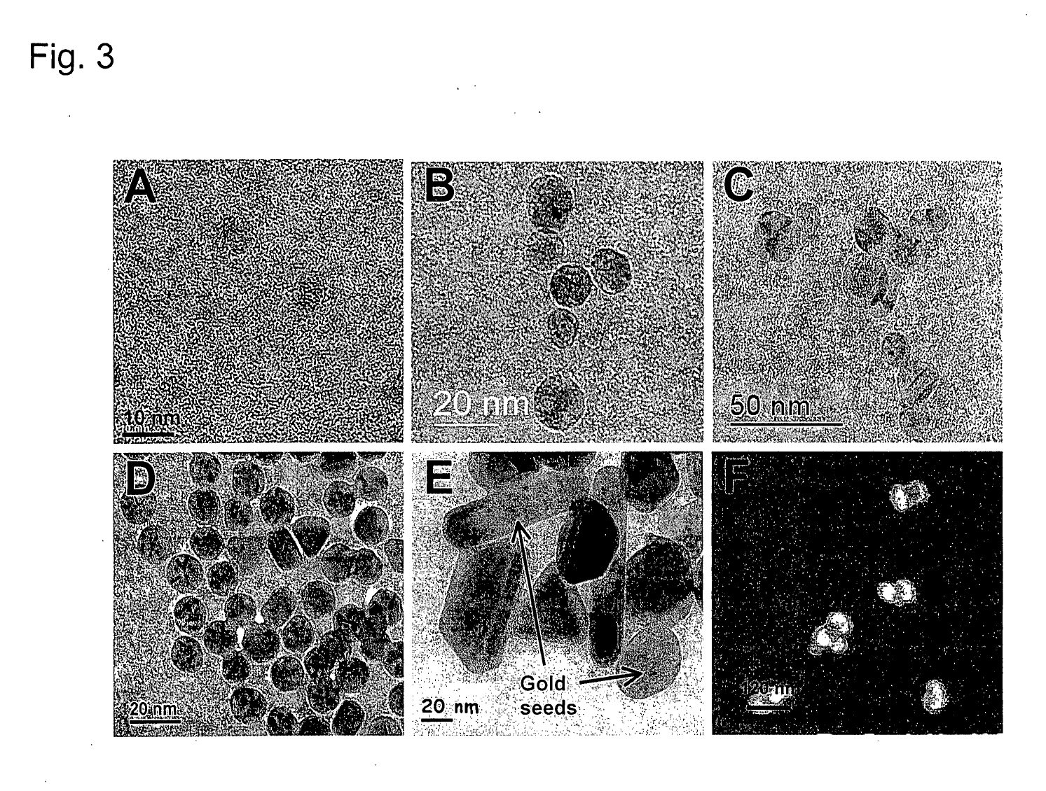

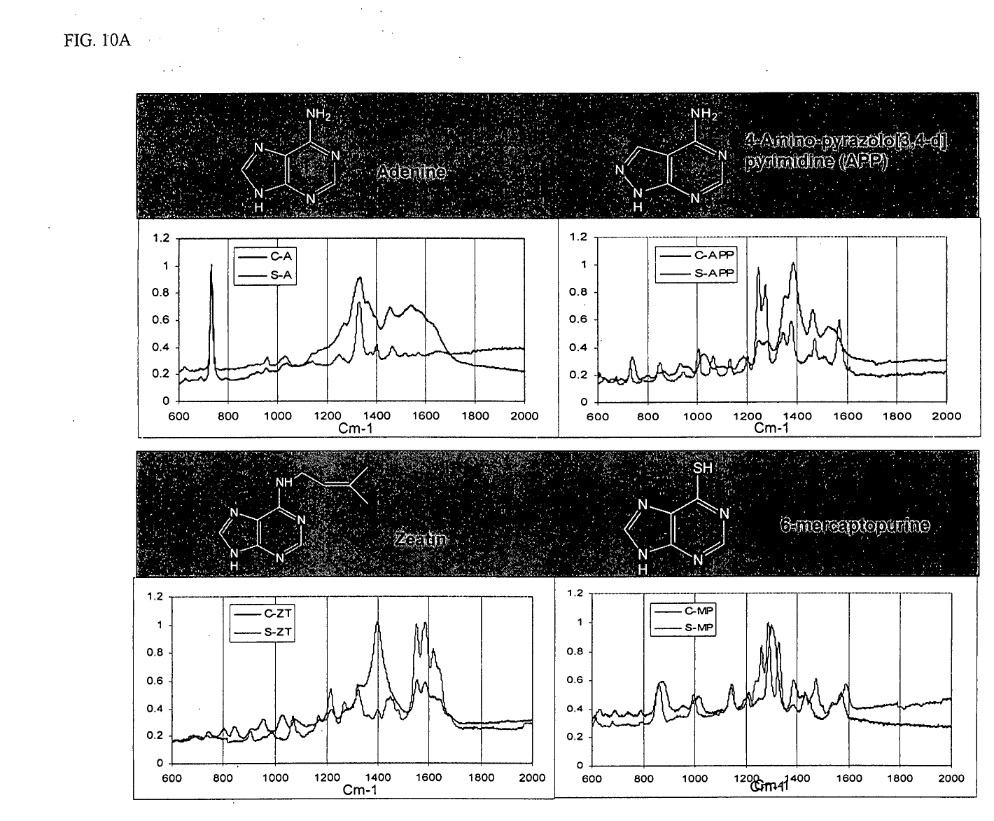

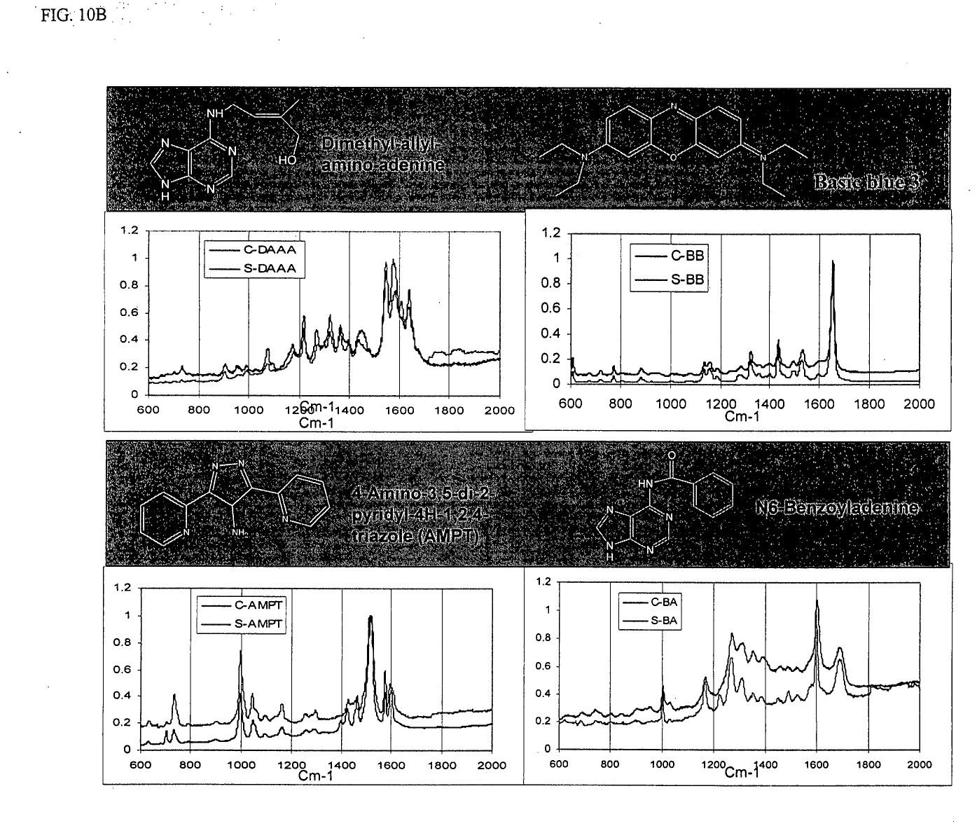

The above experimental results also suggest strongly that the Raman label molecules associated with the COIN particles interacted with silver metal in a way that was different from simple adsorption as in SERS. Since no covalent attachment was used, the label molecules were likely embedded or trapped in the metal lattices. This could occur during COIN synthesis because Raman labels were contacted with metal nanoparticles when the nanoparticles were undergoing enlargement and clustering. Small metal particles were chosen as seeds to provide a large surface area for initial Raman label absorption, and relatively high concentrations of Raman labels wee used to induce particle aggregation by reducing surface zeta potentials of the seed particles (FIG. 9). It was found that both Raman labels and an elevated temperature (i.e., boiling) were required for rapid silver atom rearrangement as indicated by the increased sizes of silver particles (FIG. 3B). During metal particle (gold and silver) enlargement and clustering, Raman label molecules could be depleted from the solution (FIG. 10). The label depletion phenomenon was also supported by the observation that 10-20 μM of a Raman label (aza-adenine) could be used to make a stable COIN solution from 1 mM AgNO₃; but the same amount of label would cause aggregation of the same amount of silver particles with similar sizes as the COIN particles. The above information is consistent with the assertion that Raman labels are trapped or embedded in COIN particles.

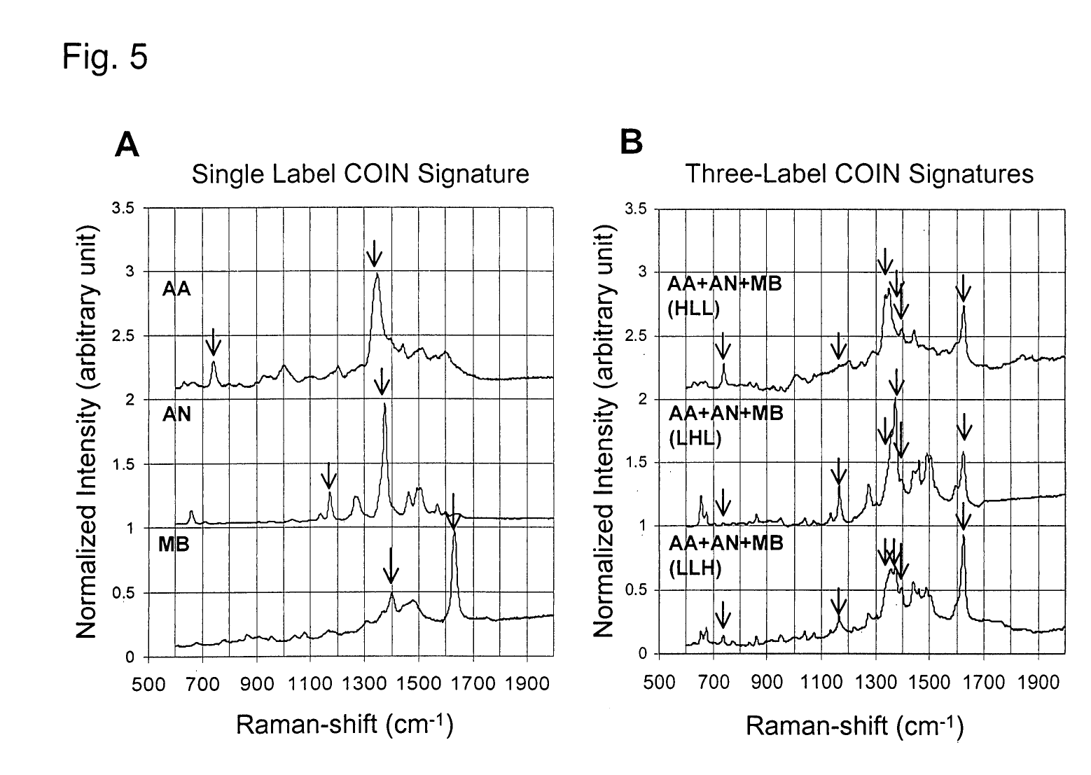

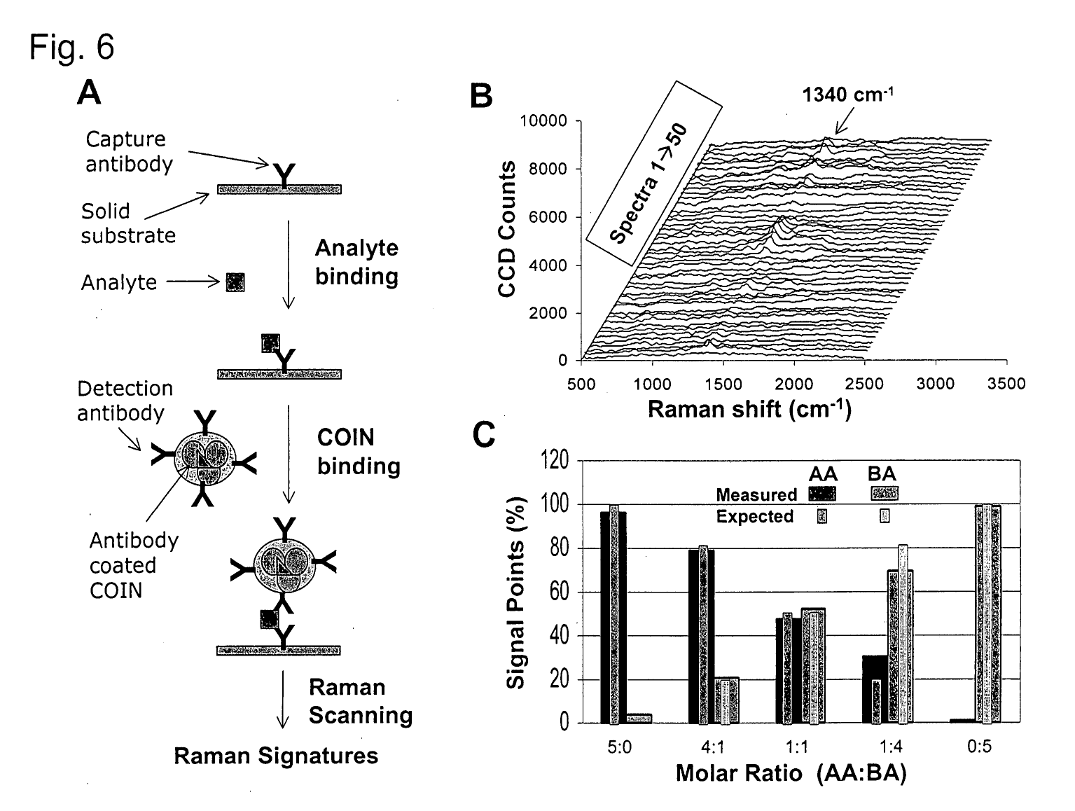

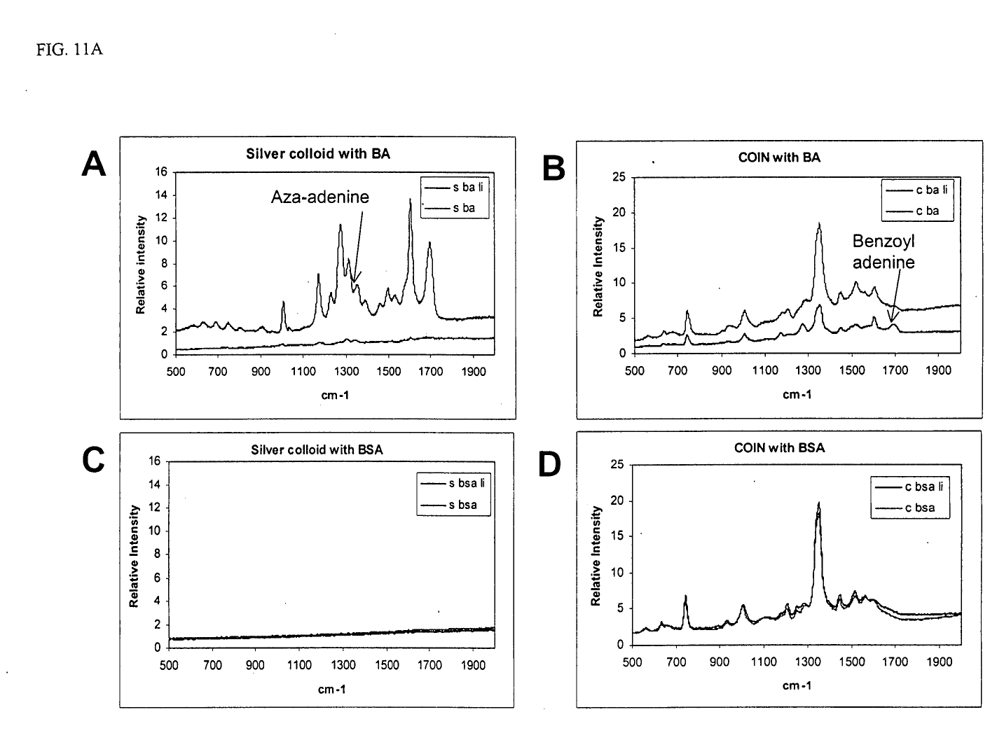

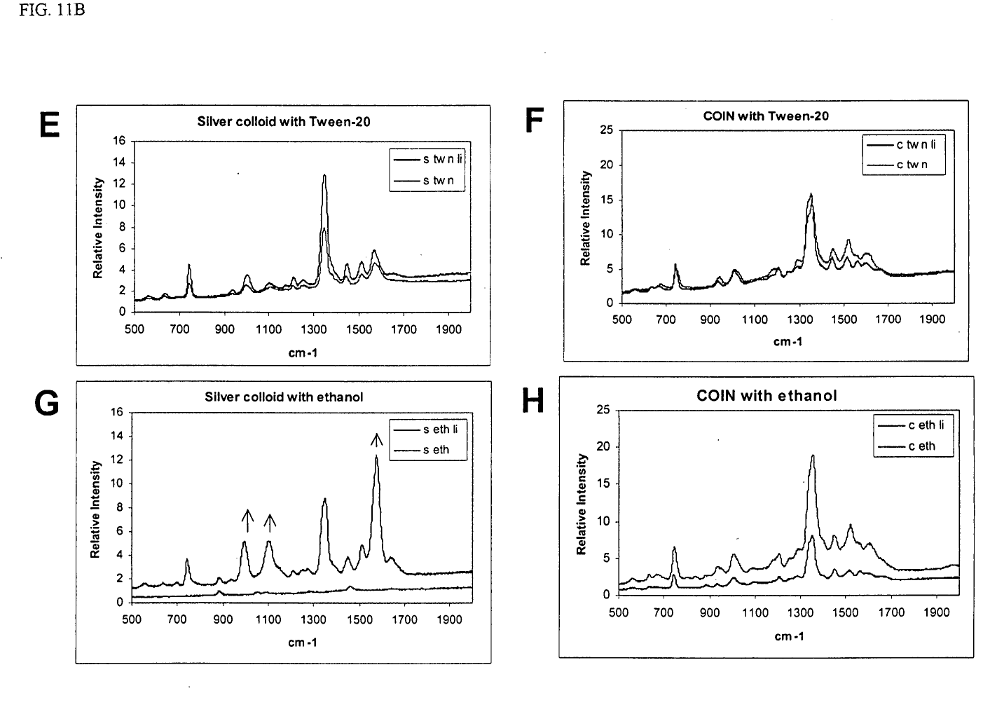

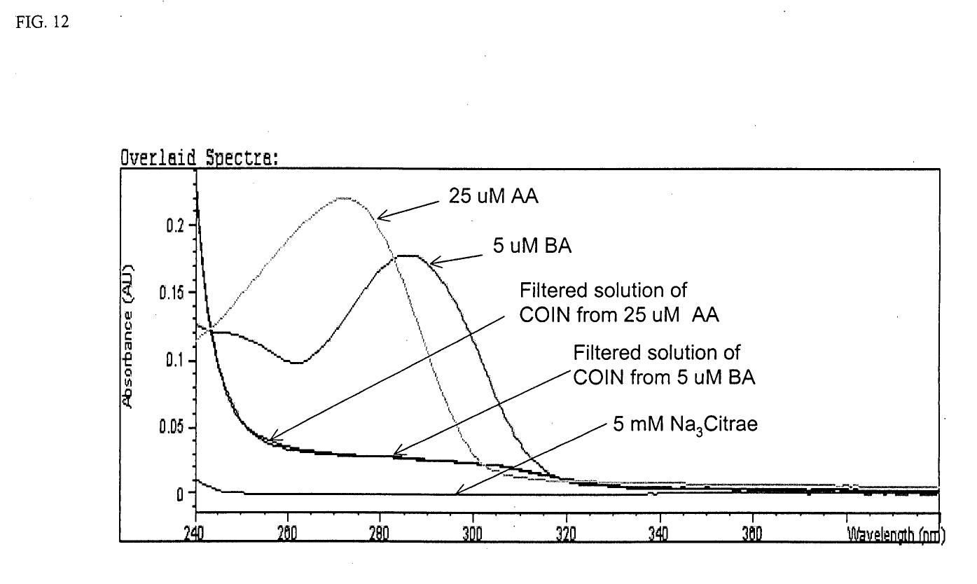

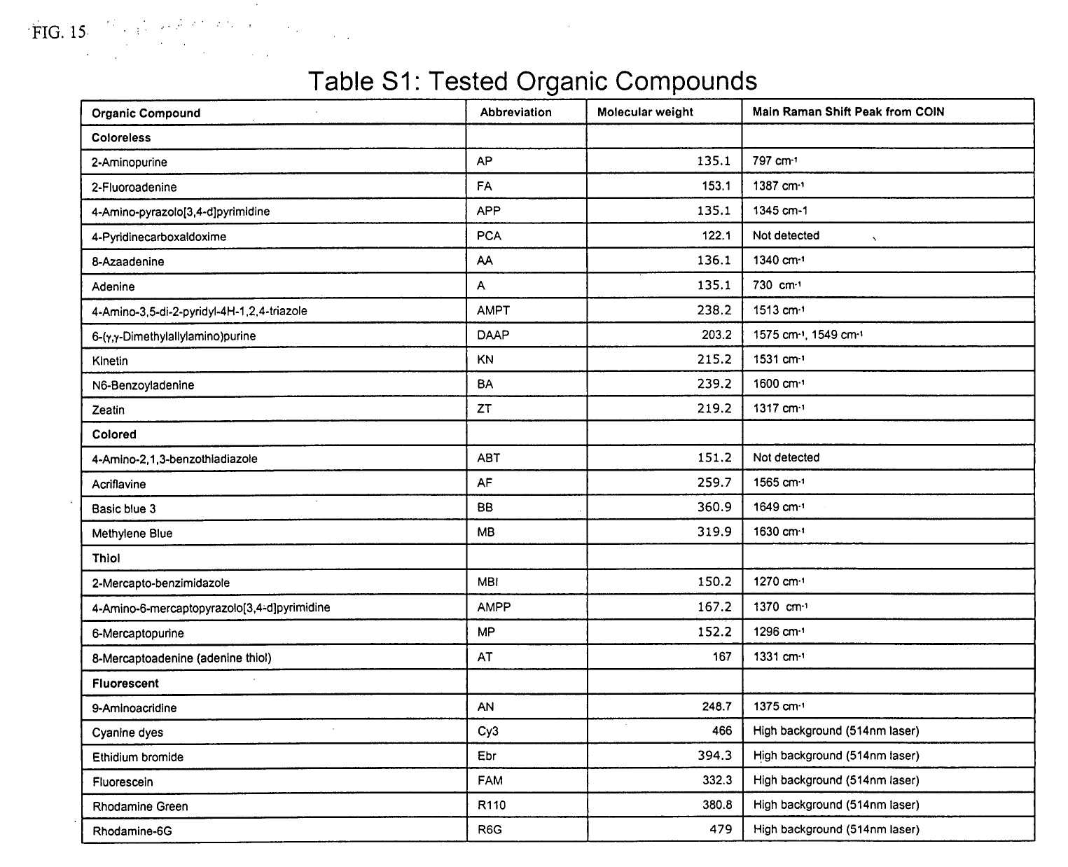

To know what types of Raman labels are compatible with COIN, various Raman labels were tested for COIN synthesis. FIG. 5A shows that aza-adenine (AA, colorless, non fluorescent), methylene blue (MB, colored dye) and amino-acridine (AN, fluorescent dye) produced COINs with unique signatures. For 9 of the colorless and non-fluorescent compounds (mostly adenine-derivatives) that have strong Raman signals in SERS, 7 of them were incorporated in COINs in the first or second trials (FIG. 15 and FIG. 11). Those that did not produce active COIN were compounds that had active thiol groups. It was likely that the thiol group reacted with the metal surface and thus created an effect that inhibited active COIN formation. To the contrary, other approaches used only Raman labels that had active thiol groups. For all the colored or fluorescent compounds, almost all were incorporated in active COINs (FIG. 15). Based on these data, it is concluded that many other commercial available Raman active labels are compatible with the COIN technique. Considering that many Raman-active compounds can be synthesized without the requirement of providing reactive groups for covalent attachment, it is believed that the availability of Raman labels for this approach is unlimited.

Data presented so far also suggested that the label-metal interaction is mutually beneficial. Besides serving as signal sources, organic label molecules promote particle enlargement, clustering and even surface defects (FIG. 3C), all of which are in favor of EME. On the other hand, the metal atoms or the metal crystal structures provide spaces to hold and stabilize Raman label molecules, especially those in the cluster junctions.

### Example 4

### Numerous Raman Signatures Can Be Created

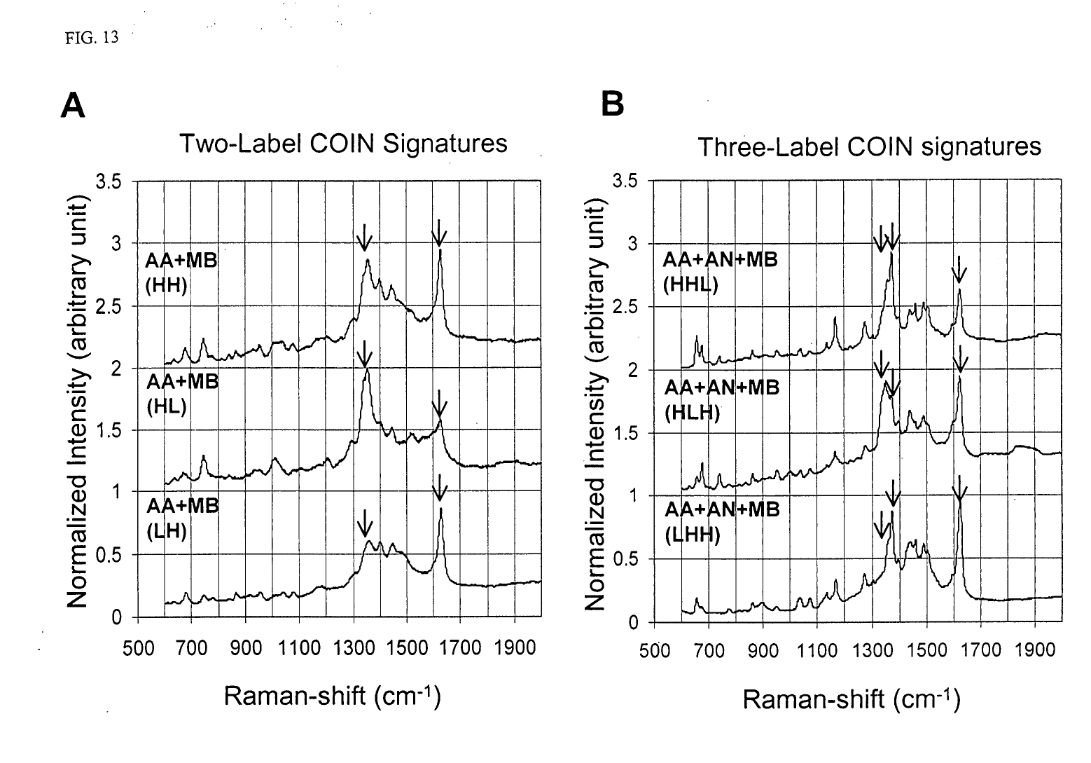

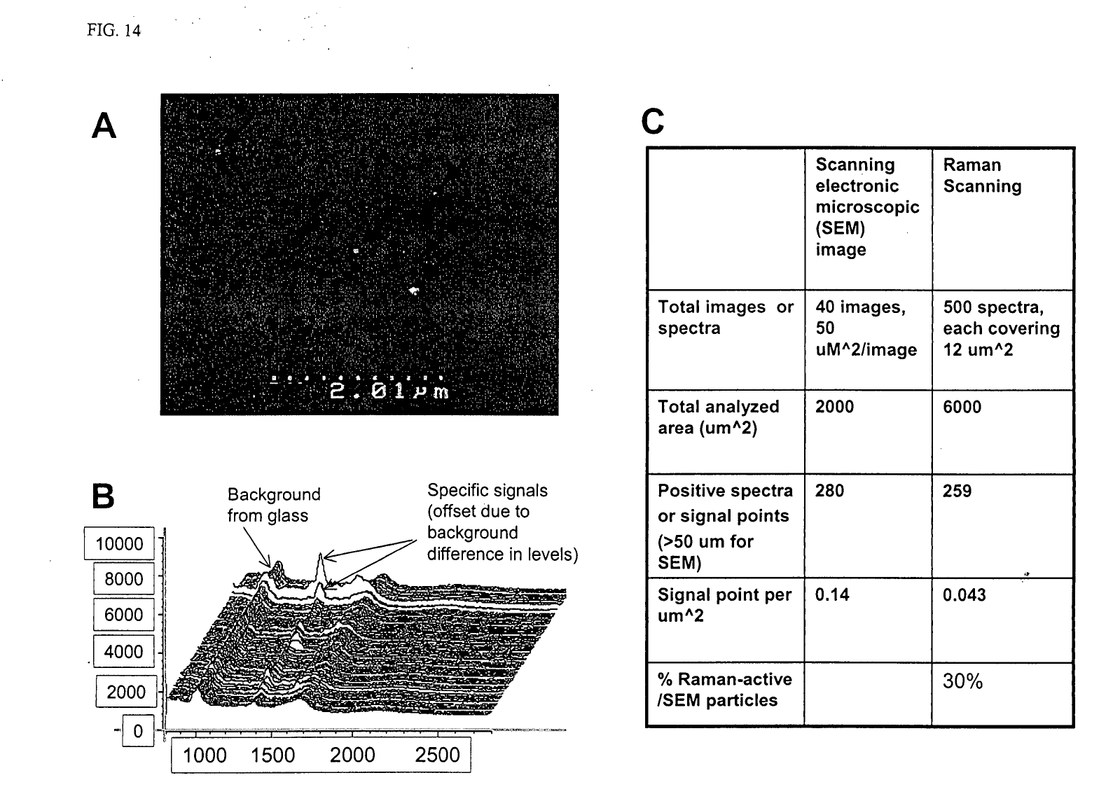

Using the OCAMF-based COIN synthesis chemistry, it is possible to generate a large number of different COIN signatures by mixing a limited number of Raman labels. In a simplified scenario, the Raman spectrum of a COIN sample can be characterized by at least three parameters: (a) peak position, depending on the chemical structure of Raman labels used and the umber of available labels (designated as L), (b) peak number, depending on the number of labels mixed in a COIN (designated as M), and (c) peak height, depending on ranges of relative peak intensity (designated as i), which is governed by, but not necessarily proportional to, the label concentrations. The total number of possible Raman signatures (designated as T) can be calculated from the following equation:

$$T = {\sum }_{k = 1}^{M} \frac{L !}{( L - k ) ! k !} P ( i , k )$$

where P(i, k)=i^{k}−i+1, being the intensity multiplier which represents the number of distinct Raman spectra that can be generated by combining k (k=1 to M) labels for a given i value. To demonstrate that multiple labels can be mixed to make COINs, we tested the combinations of 3 Raman labels for COIN synthesis (L=3, M=3, and I=2). As shown in FIG. 5 and FIG. 13, the results for 1 label, 2 labels and 3 labels were all as expected (total 18 distinguishable signatures expected, 12 shown, 6 not shown were from 2 label combinations). These spectral signatures demonstrated that closely positioned peaks (15 cm⁻¹ between AA and AN) could be resolved visually. In practical applications, mathematical and statistical methods can be used for signature reorganization.

Theoretically, over a million of COIN signatures could be made within the Raman shift range of 500-2000 cm⁻¹. With about a dozen Raman labels that had been tested (say, L=13), it is possible to generate near 2000 COIN signatures (M=3, i=2). When increasing the value of L to about 30 (Supplementary Data), which is achievable, the possible COIN number would be 50,000, equivalent to the number of genes in the human genome.

When known Raman labels are used, signature recognition can be achieved by comparing detected spectra with spectra that are theoretically predicted. When COINs are mixed and detected, a standard deconvolution algorithm can be used to resolve the signatures. To avoid data analysis complications, experimental designs and experimental conditions may be adjusted so that mixed signatures are minimized.

### Example 5

### Conjugation of COIN Particles with Detection Antibodies

A 500 uL solution containing 2 ng of a biotinylated anti-human antibody (anti-IL-2 or anti-IL-8) in 1 mM sodium citrate (pH 9) was mixed with 500 uL of a COIN solution (made with 8-aza-adenine or N-benzoyl-adenine); the resulting solution was incubated at room temperature for 1 hour, followed by adding 100 uL of PEG-400. The solution was incubated at room temperature for another 30 min, then 200 uL of 1% Tween-20 was added to the solution. The solution was centrifuged at 2000×g for 10 min. After removing the supernatant, the pellet was resuspended in 1 mL solution (BSAT) containing 0.5% BSA, 0.1% Tween-20 and 1 mM sodium citrate. The solution was then centrifuged at 1000×g for 10 min. The BSAT washing procedure was repeated for a total of 3 times. The final pellet was resuspended in 700 uL of diluting solution (0.5% BSA, 1×PBS, 0.05% Tween-20). The Raman activity of COIN was measured and adjusted to a specific activity of about 500 photon counts per ul per 10 seconds using a Raman spectroscope that generated about 600 counts from methanol at 1040 cm⁻¹ for 10 second collection time.

Confirmation of antibody-COIN conjugation: To obtain a standard curve, ELISA experiments were performed according to manufacture's instruction (BD Bioscences), using immobilized capture antibody, fixed analyte concentration (5 ng/mL IL-2 protein) and a serially diluted detection antibody (0, 0.01, 0.1, 1, and 10 ug/mL). After detection antibody binding, streptavidin-HRP (Horse Radish Peroxidase) was then reacted with the biotinylated detection antibodies and TMB (Tetramethyl Benzidine) substrate was applied followed by UV absorption measurement. Standard curve was generated by plotting absorption values against antibody concentrations.

To estimate the amount of antibody molecules that could be attached to a COIN particle, similar ELISA experiment was then performed with COIN-biotinylated Anti-IL2 detection antibody. The ELISA data were collected and the binding activity of the COIN-antibody conjugate was compared with the standard curve to estimate the equivalent amount of antibody in the COIN-antibody conjugate. Assuming that only one of the antibody molecules that had been conjugated to a COIN particle bound to an immobilized analyte, and that all biotin moieties associated with the COIN particle were bound by streptavidin-HRP. Finally, the number of antibody molecules per COIN was estimated by dividing the equivalent amount of antibody in the COIN-antibody by the estimated number of COIN particle.

Immuno assays: (1) Assay support Preparation: Xenobind™ Aldehyde slide (Polysciences, Inc., PA, USA) were used as substrates for immuno assays; before being used, wells on a slide were prepared by overlaying a piece of cured PDMS of 1 mm thick (ref.), which had wholes of 5 mm in diameter. (2) Capture antibody binding: Anti-Human IL2 antibody (9 ug/mL) was prepared in 0.33×PBS; 50 uL of the antibody was added to a well on the slide and the slide was incubated in a humidity chamber at 37° C. for 2 hours. (3) Surface blocking: After removing antibody solution, 50 uL of 1% BSA in a 10 mM glycine solution was added to each well to quench the aldehyde groups. The slide was incubated at 37° C. for another 1 hour, then the wells were washed 4 times, each with 50 uL PBST washing solution containing 1×PBS, 0.05% Tween-20. (4). Protein binding: IL-2 and IL-8 solutions at various concentrations (from 0-50 ug/mL, depending on experiments) were prepared in ELISA diluting solution (1×PBS, 0.5% BSA, 0.05% Tween-20). A sample containing 40 uL of an antibody solution was added to a well; binding was carried out at 37° C. from several hours (over night was preferred to ensure binding was complete). The sample-containing wells were washed with 50 uL of PBST solution for a total of 4 times. (5) Detection antibody binding: equal amounts of COIN samples conjugated with anti-IL2 detection antibody and anti-IL8 detection antibody, respectively, were combined and then added to each PDMS well; the solutions were then incubated at 37° C. for 1 hour. After removing the conjugate solutions, the wells were washed four times, each with 50 uL of ELISA Wash Solution, followed by washing with 50 uL of DI-water once. Finally, 30 uL of DI-water was added to each well before SERS measurement.

Although the invention has been described with reference to the above example, it will be understood that modifications and variations are encompassed within the spirit and scope of the invention. Accordingly, the invention is limited only by the following claims.

1. A composite organic-inorganic nanoparticle comprising a core and a surface, wherein the core comprises a metallic particle comprising a first metal and at least one organic compound.

2. The nanoparticle of claim 1, wherein the organic compound is a Raman-active organic compound.

3. The nanoparticle of claim 1, further comprising a second metal different from the first metal, wherein the second metal forms a layer overlying the surface of the nanoparticle.

4. The nanoparticle of claim 1 or 3, further comprising an organic layer overlying the metal layer.

5. The nanoparticle of claim 4, wherein the organic layer is covalently bound to the metal layer.

6. The nanoparticle of claim 4, wherein the organic layer comprises a probe.

7. The nanoparticle of claim 6, wherein the probe is selected from antibodies, antigens, polynucleotides, oligonucleotides, receptors, peptide nucleic acids (PNA), carbohydrates, or ligands.

8. The nanoparticle of claim 6, wherein the probe is a polynucleotide.

9. The nanoparticle of claim 8, wherein the polynucleotide is DNA or RNA.

10. The nanoparticle of claim 1, wherein the metallic particle comprises gold, silver, platinum, copper, or aluminum.

11. The nanoparticle of claim 1, wherein the metallic particle comprises gold.

12. The nanoparticle of claim 1, wherein the metallic particle comprises metal oxides.

13. The nanoparticle of claim 12, wherein the metallic particle comprises iron oxides.

14. The nanoparticle of claim 3, wherein the metal layer comprises silver, gold, platinum, copper, or aluminum.

15. The nanoparticle of claim 3, wherein the metal layer comprises silver.

16. The nanoparticle of claim 1, wherein the organic compound comprises at least one nitrogen atom and at least one aromatic ring.

17. The nanoparticle of claim 1, wherein the organic compound is a nitrogen-containing aromatic heterocycle.

18. The nanoparticle of claim 1, wherein the organic compound is adenine or an analog thereof.

19. The nanoparticle of claim 1, wherein the organic compound is adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine.

20. The nanoparticle of claim 1, further comprising a fluorescent label.

21. The nanoparticle of claim 1, wherein the Raman-active organic compound has a molecular weight less than about 500 Daltons.

22. The nanoparticles of claim 1, wherein the nanoparticles are substantially spherical.

23. The nanoparticles of claim 1, wherein the nanoparticles have a diameter about 20 nm up to 60 nm.

24. The nanoparticles of claim 1, wherein the nanoparticles are aggregated to form clusters ranging from about 50 nm to 200 nm.

25. A method for producing a composite organic-inorganic nanoparticle having a core and a surface, comprising:
reducing metallic ions in the presence of a Raman-active organic compound under conditions suitable to form a metallic colloid,
thereby producing a composite organic-inorganic nanoparticle having a core and a surface, wherein the core comprises the metallic colloid and the Raman-active organic compound.

26. The method of claim 24, further comprising forming a metallic layer overlying the surface of the nanoparticle, wherein the metallic layer comprises a second metal different from the metallic colloid.

27. The method of claim 26, wherein the metallic layer is formed by contacting the nanoparticle with a plurality of second metal ions under conditions suitable to reduce the second metal ions.

28. The method of claim 26, further comprising forming an organic layer overlying the metallic layer, wherein the organic layer is formed by contacting the nanoparticle with an organic compound under conditions suitable to form covalent linkages between the metallic layer and the organic compound.

29. The method of claim 25, 26 or 28, further comprising incorporation of a magnetic composition.

30. The method of claim 29, wherein the magnetic composition is an iron oxide.

31. A method for producing a composite organic-inorganic nanoparticle having a core and a surface, comprising
vapor-depositing a metal in the presence of a Raman-active organic compound under conditions suitable to form metallic nanoparticles, thereby producing a composite organic-inorganic nanoparticle having a core and a surface, wherein the core comprises metallic colloids and the Raman-active organic compound.

32. A method for producing a composite organic-inorganic nanoparticle having a core and a surface, comprising
etching a metal surface in the presence of a Raman-active organic compound under conditions suitable to form metallic nanoparticles, thereby producing a composite organic-inorganic nanoparticle having a core and a surface, wherein the core comprises metallic colloids and the Raman-active organic compound.

33. A method for detecting an analyte in a sample comprising:
contacting a sample containing an analyte with a nanoparticle of claim 6, wherein the probe binds to the analyte; and
detecting SERS signals emitted by the nanoparticle, wherein the signals are indicative of the presence of an analyte.

34. The method of claim 33, wherein the analyte is a biological agent.

35. The method of claim 33, wherein the analyte is a microorganism.

36. The method of claim 35, wherein the microorganism is a virus or a bacterium.

37. The method of claim 34, wherein the biological agent is a peptide, polypeptide, antibody, protein, or a polynucleotide.

38. The method of claim 34, wherein the biological agent is a polynucleotide.

39. The method of claim 33, wherein the biological agent is DNA, RNA, or peptide nucleic acid (PNA).

40. The method of claim 33, wherein the sample is an air sample.

41. The method of claim 33, wherein the sample is a liquid sample.

42. The method of claim 33, wherein the sample is a biological sample.

43. The method of claim 33, wherein the analyte is detected in an application which is a member selected from the group consisting of environmental toxicology, remediation, biomedicine, material quality control, food monitoring, agricultural monitoring, heavy industrial manufacturing, ambient air monitoring, worker protection, emissions control, product quality testing, oil/gas petrochemical applications, combustible gas detection, H₂S monitoring, hazardous leak detection, emergency response and law enforcement applications, explosives detection, utility and power applications, food/beverage/agriculture applications, freshness detection, fruit ripening control, fermentation process monitoring and control, flavor composition and identification, product quality and identification, refrigerant and fumigant detection, cosmetic/perfume applications, fragrance formulation, chemical/plastics/pharmaceuticals applications, fugitive emission identification, solvent recovery effectiveness, hospital/medical applications, anesthesia and sterilization gas detection, infectious disease detection, breath analysis and body fluids analysis.

44. A system for detecting an analyte in a sample comprising:
an array comprising more than one nanoparticle of claim 1;
a sample containing at least one analyte;
a Raman spectrometer; and
a computer comprising an algorithm for analysis of the sample.

45. A kit for labeling composite organic-inorganic nanoparticles comprising
a plurality of nanoparticles of claim 1, 2, or 6, and
a biological agent.

46. The kit of claim 45, wherein the biological agent is a peptide, polypeptide, protein, antibody, or a polynucleotide.

47. The kit of claim 45, wherein the biological agent is a polynucleotide.

48. The kit of claim 45, wherein the biological agent is DNA, RNA or peptide nucleic acid (PNA).

49. A method of identifying a microorganism comprising:
contacting a sample suspected of containing the microorganism with an array of nanoparticles of claim 1;
detecting SERS signals upon contacting the sample with the nanoparticles; and
associating the SERS signals from the nanoparticles with the identity of the microorganism.

50. A method of identifying an analyte comprising:
contacting a sample suspected of containing the analyte with an array of nanoparticles of claim 1;
detecting SERS signals upon contacting the sample with the nanoparticles; and
associating the SERS signals from the nanoparticles with the identity of the analyte.
detecting SERS signals upon contacting the sample with the nanoparticles; and
associating the SERS signals from the nanoparticles with the identity of the microorganism.

51. A microsophere comprising a polymeric bead and a plurality of nanoparticles of claim 1, wherein the nanoparticles are embedded within the polymeric bead.

52. The microsphere of claim 51, wherein the polymeric bead comprises a polyolefin.

53. The microsphere of claim 51, wherein the polymeric bead comprises polystyrene.

54. The microsphere of claim 51, wherein the polymeric bead comprises a polyacrylate.

55. The microsphere of claim 51, wherein the polymeric bead comprises a poly(meth)acrylate.
